SEQUENCE LISTING (54 sequences)

SEQ ID NO: 1 - DNA, 120 bp, Cytomegalovirus
  source 1..120: mol_type = other DNA
taggcgtagtcggtgggaggtctatataagcagagctcgtttagtgaaccgtcagatcgcctggagacgccatccacgctgttttgacctccatagaagacaccgggaccgatccagcct

SEQ ID NO: 2 - DNA, 23 bp, Synthetic construct
  source 1..23: mol_type = other DNA
gctagcgcctcgactgtgccttc

SEQ ID NO: 3 - DNA, 30 bp, Synthetic construct
  source 1..30: mol_type = other DNA
gctagcctcgagctcagaagccatagagcc

SEQ ID NO: 4 - DNA, 22 bp, synthetic construct
  source 1..22: mol_type = other DNA
acgcgttcgccaccatggtgag

SEQ ID NO: 5 - DNA, 34 bp, Synthetic construct
  source 1..34: mol_type = other DNA
acgcgtgctagcttacttgtacagctcgtccatg

SEQ ID NO: 6 - DNA, 22 bp, Synthetic construct
  source 1..22: mol_type = other DNA
gatatctaggcgtgtacggtgg

SEQ ID NO: 7 - DNA, 86 bp, Synthetic construct
  source 1..86: mol_type = other DNA
acgcgtaggctggatcggtcccggtgtcttctatggaggtcaaaacagcgtggatggcgtctccaggcgatctgacggttcactaa

SEQ ID NO: 8 - DNA, 31 bp, Synthetic construct
  source 1..31: mol_type = other DNA
gttaacgtttaaacgggcagagcgcacatcg

SEQ ID NO: 9 - DNA, 31 bp, Synthetic construct
  source 1..31: mol_type = other DNA
ccaacgcgttggctgtgttctggcggcaaac

SEQ ID NO: 10 - DNA, 25 bp, Synthetic construct
  source 1..25: mol_type = other DNA
gatatcgttagggtgtggaaagtcc

SEQ ID NO: 11 - DNA, 23 bp, Synthetic construct
  source 1..23: mol_type = other DNA
acgcgtggaatagctcagaggcc

SEQ ID NO: 12 - DNA, 24 bp, Synthetic construct
  source 1..24: mol_type = other DNA
gtttaaactaggcgtgtacggtgg

SEQ ID NO: 13 - DNA, 21 bp, Synthetic construct
  source 1..21: mol_type = other DNA
gatatcaggctggatcggtcc

SEQ ID NO: 14 - DNA, 34 bp, Synthetic construct
  source 1..34: mol_type = other DNA
agtcggtgggaggaccggtgaagcagagctcgtt

SEQ ID NO: 15 - DNA, 34 bp, Synthetic construct
  source 1..34: mol_type = other DNA
aacgagctctgcttcaccggtcctcccaccgact

SEQ ID NO: 16 - DNA, 34 bp, Synthetic construct
  source 1..34: mol_type = other DNA
catccacgctgtttgcgttaccatagaagacacc

SEQ ID NO: 17 - DNA, 34 bp, Synthetic construct
  source 1..34: mol_type = other DNA
gtgtcttctatggtaacgcaaacagcgtggatgg

SEQ ID NO: 18 - DNA, 43 bp, Synthetic construct
  source 1..43: mol_type = other DNA
atcaccggtggatccggtacccgggagcgcttctagactgcag

SEQ ID NO: 19 - DNA, 47 bp, Synthetic construct
  source 1..47: mol_type = other DNA
tcgactgcagtctagaagcgctcccgggtaccggatccaccggtgat

SEQ ID NO: 20 - DNA, 18 bp, Synthetic construct
  source 1..18: mol_type = other DNA
acggatctcgacggtatc

SEQ ID NO: 21 - DNA, 20 bp, Synthetic construct
  source 1..20: mol_type = other DNA
agccatacgggaagcaatag

SEQ ID NO: 22 - DNA, 32 bp, Synthetic construct
  source 1..32: mol_type = other DNA
gtggatccggtacccgggaattggctccggtg

SEQ ID NO: 23 - DNA, 37 bp, Synthetic construct
  source 1..37: mol_type = other DNA
tctagaagcgctcccttacttgtacagctcgtccatg

SEQ ID NO: 24 - DNA, 21 bp, Synthetic construct
  source 1..21: mol_type = other DNA
cactacctgagcacccagtcc

SEQ ID NO: 25 - DNA, 20 bp, Synthetic construct
  source 1..20: mol_type = other DNA
gtctccaggcgatctgacgg

SEQ ID NO: 26 - DNA, 35 bp, Synthetic construct
  source 1..35: mol_type = other DNA
gtacaagtaagggagcgctcgactgtgccttctag

SEQ ID NO: 27 - DNA, 43 bp, Synthetic construct
  source 1..43: mol_type = other DNA
gaggttgattgtcgagctagcgatatctcagaagccatagagc

SEQ ID NO: 28 - DNA, 19 bp, Synthetic construct
  source 1..19: mol_type = other DNA
gtccagctcgaccaggatg

SEQ ID NO: 29 - DNA, 60 bp, Cytomegalovirus
  source 1..60: mol_type = other DNA
taggcgtagtcggtgggaggtctatataagcagagctcgtttagtgaaccgtcagatcgc

SEQ ID NO: 30 - DNA, 60 bp, Cytomegalovirus
  source 1..60: mol_type = other DNA
ctggagacgccatccacgctgttttgacctccatagaagacaccgggaccgatccagcct

SEQ ID NO: 31 - DNA, 18 bp, Cytomegalovirus
  source 1..18: mol_type = other DNA
gaggtctatataagcaga

SEQ ID NO: 32 - DNA, 18 bp, Cytomegalovirus
  source 1..18: mol_type = other DNA
gaggaccggtgaagcaga

SEQ ID NO: 33 - DNA, 18 bp, Cytomegalovirus
  source 1..18: mol_type = other DNA
ctgttttgacctccatag

SEQ ID NO: 34 - DNA, 18 bp, Cytomegalovirus
  source 1..18: mol_type = other DNA
ctgtttgcgttaccatag

SEQ ID NO: 35 - DNA, 22 bp, Synthetic construct
  source 1..22: mol_type = other DNA
gcttcaccagatggctgtgata

SEQ ID NO: 36 - DNA, 21 bp, Synthetic construct
  source 1..21: mol_type = other DNA
aataagtggtgccgtcccttg

SEQ ID NO: 37 - DNA, 20 bp, Synthetic construct
  source 1..20: mol_type = other DNA
agaaggcctatgagctgtcc

SEQ ID NO: 38 - DNA, 20 bp, Synthetic construct
  source 1..20: mol_type = other DNA
ttgccggtctcactggtgat

SEQ ID NO: 39 - DNA, 21 bp, Synthetic construct
  source 1..21: mol_type = other DNA
caagacaaatcatccggctac

SEQ ID NO: 40 - DNA, 21 bp, Synthetic construct
  source 1..21: mol_type = other DNA
gtacttgcccagctggcagag

SEQ ID NO: 41 - DNA, 21 bp, Synthetic construct
  source 1..21: mol_type = other DNA
tgaagcccaccagagccccct

SEQ ID NO: 42 - DNA, 21 bp, Synthetic construct
  source 1..21: mol_type = other DNA
gatgctgcggcggaagaagcc

SEQ ID NO: 43 - DNA, 20 bp, Synthetic construct
  source 1..20: mol_type = other DNA
gcatggtgctactgtgtaag

SEQ ID NO: 44 - DNA, 20 bp, Synthetic construct
  source 1..20: mol_type = other DNA
gcacagcatctctagacatg

SEQ ID NO: 45 - DNA, 21 bp, Synthetic construct
  source 1..21: mol_type = other DNA
tggactccaacaacaacacag

SEQ ID NO: 46 - DNA, 20 bp, Synthetic construct
  source 1..20: mol_type = other DNA
gatggtgggaagtaggtggg

SEQ ID NO: 47 - DNA, 21 bp, Synthetic construct
  source 1..21: mol_type = other DNA
acagtccatgccatcactgcc

SEQ ID NO: 48 - DNA, 21 bp, Synthetic construct
  source 1..21: mol_type = other DNA
gcctgcttcaccaccttcttg

SEQ ID NO: 49 - DNA, 19 bp, Synthetic construct
  source 1..19: mol_type = other DNA
cgacagtcagccgcatctt

SEQ ID NO: 50 - DNA, 18 bp, Synthetic construct
  source 1..18: mol_type = other DNA
ccccatggtgtctgagcg

SEQ ID NO: 51 - DNA, 10 bp, Cytomegalovirus
  source 1..10: mol_type = other DNA
tctatataag

SEQ ID NO: 52 - DNA, 10 bp, Cytomegalovirus
  source 1..10: mol_type = other DNA
cttatataga

SEQ ID NO: 53 - DNA, 10 bp, Cytomegalovirus
  source 1..10: mol_type = other DNA
tttgacctcc

SEQ ID NO: 54 - DNA, 10 bp, Cytomegalovirus
  source 1..10: mol_type = other DNA
ggaggtcaaa

(19) United States
(12) Patent Application Publication
(10) Pub. No.: US 2024/0148792 A1
(43) Pub. Date: May 9, 2024

(54) VECTOR SYSTEMS AND METHODS OF USING SAME

(71) Applicant: Children's Hospital Medical Center, Cincinnati, OH (US)

(72) Inventor: Dao Pan, Cincinnati, OH (US)

(21) Appl. No.: 18/224,735

(22) Filed: Jul. 21, 2023

Related U.S. Application Data
(60) Provisional application No. 63/390,981, filed on Jul. 21, 2022.

(51) Int. Cl. A61K 35/28; A61K 48/00; C12N 9/22; C12N 15/11; C12N 15/113; C12N 15/86

(52) U.S. Cl. CPC A61K 35/28; A61K 48/005; C12N 9/22; C12N 15/111; C12N 15/113; C12N 15/86; C12N 2310/20; C12N 2710/16143

(57) ABSTRACT

Disclosed are delivery vectors for the delivery of nucleic acid transcription products and methods of using same. The disclosed vectors may be either contain a single transcription unit (TU) (“single-cassette”) or two transcription units (TUs) (“dual-cassette”). The delivery vectors employ a cytomegalovirus (CMV) core fragment (cC element) that may be oriented in either a forward or reverse orientation to improve vector transcription efficiency, which is located downstream of a TU in a single-cassette system, or placed between two TUs in a dual cassette system. The disclosed delivery vectors may be used for the delivery of therapeutic or non-therapeutic proteins, peptides, or non-coding, and may be used both in vitro and in vivo.

### CROSS-REFERENCE TO RELATED APPLICATIONS

This application claims priority to and benefit of U.S. Provisional Application Ser. No. 63/390,981, filed Jul. 21, 2022, the contents of which are incorporated in their entirety for all purposes.

### STATEMENT REGARDING FEDERALLY-SPONSORED RESEARCH

This invention was made with government support under NS086134 and NS064330 awarded by the National Institutes of Health. The government has certain rights in the invention.

### REFERENCE TO SEQUENCE LISTING

A Sequence Listing submitted as an XML file is hereby incorporated by reference. The name of the XML file for the Sequence Listing is 2023-1204-SL 18224735.xml, the date of creation of the XML file is Dec. 4, 2023, and the size of the ASCII text file is 48,886 bytes.

### BACKGROUND

Co-expression of multiple genes in single vectors has achieved varying degrees of success by employing two promoters and/or application of viral 2A-peptide or the internal ribosome entry-site (IRES). However, promoter interference, potential functional-interruption of expressed-proteins by 2A-generated residual peptides or weaker translation of IRES-mediated downstream genes has curtailed their utilization. Thus, there is the need for single vectors that robustly express multiple proteins for enhanced gene therapy applications.

### BRIEF SUMMARY

Disclosed are delivery vectors for the delivery of nucleic acid transcription products and methods of using same. The disclosed vectors may be either contain a single transcription unit (TU) (“single-cassette”) or two transcription units (TUs) (“dual-cassette”). The delivery vectors employ a cytomegalovirus (CMV) core fragment (cC element) that may be oriented in either a forward or reverse orientation to improve vector transcription efficiency, which is located downstream of a TU in a single-cassette system, or placed between two TUs in a dual cassette system. The disclosed delivery vectors may be used for the delivery of therapeutic or non-therapeutic proteins, peptides, or non-coding RNA, and may be used both in vitro and in vivo.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A-1C. Promoter activities in dual-cassette expression are dependent on their relative strength and position. FIG. 1A. Schematic diagrams of SIN lentiviral vectors. Single-reporter LVs expressed either EGFP (GFP) from human EF-1α core promoter (Es only) or mCherry from core-CMV element (cC only). Dual-reporter LVs expressed both GFP and mCherry from Es and cC promoters. P(A)1 and p(A)2 are polyadenylation signal sequences from SV40 virus and bovine growth hormone, respectively. FIG. 1B. Representative flow cytometry dot plots showing dual-expression in various cells. FIG. 1C. Changes of median fluorescence intensity (MFI) of GFP and mCherry in stably transduced cells (<25% transduction frequency as determined by detectable reporter %), showing expression levels (transduced/untransduced cells) of Es with cC promoters in bi-directional and unidirectional designs. Red dotted lines, no expression observed. Bars represent mean±SD, with n=4-6 wells derived from 2-3 independent transduction experiments. *p<0.05, **p<0.01, ***p<0.001 by one-way ANOVA with Tukey's comparison test; ns, not significant.

FIG. 2A-2C. The cCrev element enhances both upstream and downstream promoter activities with cell type-dependence. FIG. 2A. Schematic diagrams of lentiviral vectors. Sv, SV40 promoter; cCrev, core-CMV element in reverse orientation. FIG. 2B. Representative single-channel dot plots of GFP and mCherry expression in cells with different blood lineages by flow cytometry analysis. FIG. 2C. Changes of median fluorescence intensity (MFI) of GFP and mCherry expression in stably transduced cells (<25% transduction frequency). Red dotted lines, no expression observed. Bars represent mean±SD, with n=6-12 wells derived from 2-4 independent transduction experiments. *p<0.05, **p<0.01, ***p<0.001 by one-way ANOVA with Tukey's comparison test; ns, not significant.

FIG. 3A-3B. Enhancer effects of Cc on both upstream and downstream promoters are sensitive to orientation and cell types. FIG. 3A. Schematic diagrams of dual-expression LVs. EF, full-length human EF-1α promoter containing its intron A enhancer. FIG. 3B. MFI changes for GFP and mCherry in tested cell lines (<25% transduction frequency). Bars represent mean±SD, with n=6-12 wells derived from 2-4 independent transduction experiments. * p<0.05,** p<0.01, *** p<0.001 by one-way ANOVA with Tukey's comparison test; ns, not significant.

FIG. 4A-4B. Evaluation of potential spacer effects of cC element for dual-cassette expression. FIG. 4A. Schematic diagrams of LVs expressing both GFP from Es promoter and mCherry from SV40 with cC (cCrevSv), a spacer fragment (Sp*Sv) or SV40 promoter alone. Sp* represents either a 119 bp fragment from coding sequence of ampicillin resistance gene in pGEM T-Easy vector (Sp1) or tandem repeats of Sp1 (238 bp, Sp2). FIG. 4B. Change in median fluorescence intensity (MFI) of GFP and mCherry from dual promoters in stably transduced cells (<25% transduction frequency). Bars represent mean±SD, n=6-12 wells derived from 2-4 independent transduction experiments. * p<0.05, ** p<0.01, *** p<0.001 by one-way ANOVA with Tukey's comparison test; ns, not significant.

FIG. 5A-5C. Enhancer function of cC depends on its binding sites for transcription factors. FIG. 5A. Diagram of predicted binding sites (51 and S2) and their mutated sites (m1 and m2) for transcription factors in cC element. The location of cC (−50 to +70) are indicated with reference to the transcription start site (tss) of CMV IE gene. FIG. 5B. Schematic diagrams of LVs expressing GFP from Es promoter and mCherry from either SV40 (Sv), hybrid cC-SV40 (cCrev-Sv) or its mutated variants. FIG. 5C. Changes in mean fluorescence intensity (MFI) for GFP and mCherry expression from dual promoters in cells stably transduced with different LVs (<25% transduction frequency). Bars represent mean±SD, n=6-12 wells derived from 2-4 independent transduction experiments. *p<0.05, **p<0.01, ***p<0.001 by one-way ANOVA with Tukey's comparison test; ns, not significant.

FIG. 6A-6C. Relative expression of cC element-related transcription factors in various hematopoietic cell lines. FIG. 6A. Representative dissociation curves of RT-qPCR for transcription factors SRF, RARa and NR1D1. GAPDH was used as reference gene for relative comparison. FIG. 6B-6C. Relative mRNA levels of SRF (in B, for S1 site) as well as RARa and NR1D1 (in C, for S2 site) in different cell lines. Data were derived from 3 independent reverse transcriptions, each with qPCR in triplicate, and shown as mean±SD. * p<0.05, ** p<0.01, *** p<0.001 by one-way ANOVA with Tukey's comparison test; ns, not significant.

FIG. 7 depicts the sequence of enhancer derived from CMV promoter (A) and potential binding sites for transcriptional factors (B).

FIG. 8 is a table depicting potentially relevant transcription factors (TF and mutagenesis of the cC element.

FIG. 9 is a table depicting a list of qRT-PCR primers targeting to mRNA of TFs predicted to bind to cC element.

FIG. 10 is a table showing the methods for cloning plasmids and LV vectors.

FIG. 11 is a table showing dual-cassette transgene expression from unidirectional vectors for promoter strength in stably transduced cells.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the manufacture or use of dual-cassette vectors or single-cassette vectors having a cC element as disclosed herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, a disease or disorder refers to a pathological condition in an organism resulting from, for example, infection or genetic defect, and characterized by identifiable symptoms.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The vectors disclosed herein may be used to enable long term gene expression, resulting in long term expression of a protein. As described herein, the phrases “long term expression”, “sustained expression” and “persistent expression” are used interchangeably. Long term expression according to the present invention means expression of a therapeutic gene and/or protein, preferably at therapeutic levels, for at least 45 days, at least 60 days, at least 90 days, at least 120 days, at least 180 days, at least 250 days, at least 360 days, at least 450 days, at least 720 days or more. In one aspect, long-term expression means expression for at least 90 days, at least 120 days, at least 180 days, at least 250 days, at least 360 days, at least 450 days, at least 720 days or more, or at least 360 days, at least 450 days, at least 720 days or more. Long-term expression may be achieved by repeated doses or by a single dose. In one aspect, a high level of expression may mean expression of a therapeutic gene and/or protein at a concentration of at least about 100 nM, at least about 200 nM, at least about 300 nM, at least about 400 nM, at least about 500 nM, at least about 600 nM, at least about 700 nM, at least about 800 nM, at least about 900 nM, at least about 1 μM, at least about 1.1 μM, at least about 1.2 μM, at least about 1.3 μM, at least about 1.4 μM, at least about 1.5 μM, at least about 2 μM, at least about 3 μM, at least about 4 μM, at least about 5 μM, at least about 6 μM, at least about 7 μM, at least about 8 μM, at least about 9 μM, at least about 10 μM, at least about 11 μM, at least about 12 μM, at least about 13 μM, at least about 14 μM, at least about 15 μM, at least about 20 μM, at least about 25 μM, at least about 30 μM, at least about 40 μM, at least about 50 μM, at least about 75 μM, or at least about 100 NM or more, as measured within a tissue or plasma sample. Therapeutic expression may be defined using these same values.

As used herein, the terms “nucleic acid sequence” and “polynucleotide” are used interchangeably and do not imply any length restriction. As used herein, the terms “nucleic acid” and “nucleotide” are used interchangeably. The terms “nucleic acid sequence” and “polynucleotide” embrace DNA (including cDNA) and RNA sequences. The terms “transgene” and “gene” are also used interchangeably and both terms encompass fragments or variants thereof encoding a protein or protein fragment.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 82, 84, 86, 88, 90, 92, 94, 96, 98 or 99% of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

“Sequence identity” as used herein indicates a nucleic acid sequence that has the same nucleic acid sequence as a reference sequence or has a specified percentage of nucleotides that are the same at the corresponding location within a reference sequence when the two sequences are optimally aligned. For example, a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full-length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

As used herein, the term, “therapeutic gene,” “therapeutic gene product” or “therapeutic polypeptide” refers to any heterologous protein expressed by a therapeutic gene encoded by the dual-cassette vectors disclosed herein, that ameliorates the symptoms of a disease or disorder or ameliorates the disease or disorder. Therapeutic gene products include, but are not limited to, moieties that inhibit cell growth or promote cell death, that can be activated to inhibit cell growth or promote cell death, or that activate another agent to inhibit cell growth or promote cell death. Exemplary therapeutic gene products include, for example, immune checkpoint inhibitors, cytokines, growth factors, photosensitizing agents, radionuclides, toxins, anti-metabolites, signaling modulators, anti-cancer antibodies, angiogenesis inhibitors, wild-type protein (e.g. for the replacement of a defective protein or supplementation of endogenous protein), or combinations thereof.

Co-expression of multiple genes robustly within the same transduced cells using a single vector has been needed for enhanced gene therapy applications in treating many diseases, including inherited genetic diseases and cancer (for multiple CAR expression in the same cells).

Applicant has found that a short-fragment within TATA-box region and exon 1 of human cytomegalovirus major immediate-early genes (−50 to +70 bp, cC element) not only boosted the potency of upstream short-version promoter (EFs or Es) of human elongation factor 1α (EF) to levels comparable to full-length EF in hematopoietic cells, but also blocked the suppression of downstream SV40 promoter by the Es, all with different patterns in different types of blood cells. Such lineage-restricted upregulation may be attributed to two protein-binding domains of cC and diverse expression of related factors in different cell types for enhancer and terminator activities but is not believed to be due to spacing function.

Vector-mediated delivery and expression of exogenous genes in biological systems have revolutionized bioscience research and its applications. Appropriate vector design is pivotal to the development of successful viral and non-viral expression systems in vitro and in vivo. Increasingly complex research and applications require robust and/or divergent expression of two or more transgenes for various purposes, such as co-expressing multiple chimeric antigen receptors (CAR) and/or cytokines into the same cells for superior cancer therapy, biological sample tracking/imaging, introducing multimeric proteins for industrial production of recombinant proteins. Unlike the challenges faced by co-transfecting a mixture of several vectors, bicistronic vectors, each expressing a single transcriptional unit (TU) from a promoter, utilize either an internal ribosome entry site (IRES) or viral 2A or 2A-like peptide elements to generate multiple gene products from one vector. Such vectors can ensure that all transgenes are regulated/expressed together at the same time and location. However, these systems also have limitations including the number of transgenes, weaker translation of IRES-mediated downstream gene and potential functional impairment of expressed proteins by 2A-generated residual peptides.

Alternatively, independent expression of multiple genes in two (or more) TUs from different promoters in the same vector would provide the advantage of being robustly regulated in a spatio-temporal manner. For instance, such a system can potentially improve the safety and efficacy of hematopoietic stem cell (HSC)-mediated gene therapy when utilizing dual-cassette lentiviral vectors (LV), in which one cassette expresses a selectable marker or a reporter from a ubiquitous promoter and the other cassette expresses a therapeutic transgene restricted to targeted blood cell types from a lineage-specific promoter. It could also increase the number of CARs and cytokines co-expressed in the same cells when in combination with the usage of IRES or 2A element. However, transcriptional (or promoter) interference between adjacent TUs may occur and often results in reduced expression or even complete suppression of genes in one or both genes. Several factors may contribute to the interference, including the comparative strengths and relative positioning of the promoters, susceptibility of promoter(s) to epigenetic modification and the roles of other cis-acting regulatory elements, although further elucidation remains an active area in vector design research. In addition, other regulatory elements such as insulators, terminator or pause sequences, polyadenylation (poly(A)) sequences and DNA spacer elements also play roles in transgene expression. While poly(A) sequences are believed to be essential in vector designs to ensure transcription termination and enhance nuclear transport, mRNA stability, and translation of each TU, the inclusion of other elements is based on their utmost necessity to meet vector and gene expression needs. Considerations of vector size capacity, expression configuration desired, and promoter-pair compatibility are considerations in dual-cassette expression vector design. Efforts to optimize such vector designs remain wanted to achieve effective, independently controlled expression of multiple genes for bioscience research and applications.

The instant disclosure seeks to address one or more noted deficiencies in the art.

Disclosed are delivery vectors useful for a variety of different purposes as disclosed herein, including both in vivo and in vitro, and for therapeutic and research purposes. Applicant has found that the use of a “cC” element, as described herein, surprisingly allowed for improved expression of sequences provided in a both dual-cassette and single-cassete delivery vectors.

The disclosed dual-cassette vectors may, in certain aspects, comprise a first transcription unit (TU) and a second transcription unit (TU). The first transcription unit may comprise a first promoter, a first nucleic acid sequence, and a first termination sequence. The second transcription unit (TU), located downstream from the first TU, may comprise a second promoter, a second nucleic acid sequence, and a second termination sequence. The dual-cassette vector may further comprises a cytomegalovirus core fragment (cC element), as described herein.

Vectors

In one aspect, the dual-cassette vector may be a non-viral plasmid vector, such as, for example, described in U.S. Pat. No. 9,233,174 and US20180028686In a further aspect, the dual-cassette vector may be a nanoparticle particle, for example, as described in U.S. Ser. No. 10/392,446. In a further aspect, the dual-cassette vector may be a virus-derived vector. Exemplary virus-derived vectors may include, for example, a lentiviral vector, an adenoviral vector (AdV), an adeno-associated viral vector (AAV), a retroviral vector, a herpes simplex virus vector (HSV), a vaccinia virus vector, a vesicular stomatitis virus (VSV), modified vaccinia virus Ankara (MVA), arenavirus, Sendai virus, measles virus, a poxvirus vector, and combinations thereof. Such vectors are known in the art and described in, for example, Zhao Z, Anselmo A C, Mitragotri S. Viral vector-based gene therapies in the clinic. Bioeng Transl Med. 2021 Oct. 20; 7(1):e10258. doi: 10.1002/btm2.10258. PMID: 35079633; PMCID: PMC8780015.

Promoter

The disclosed dual-cassette vectors comprise, in general, at least two promoters, each promoter being upstream to a nucleic acid sequence to be expressed and the termination sequence. The first promoter is located upstream to the first nucleic acid sequence and the first termination sequence, while the second promoter is located upstream of the second nucleic acid sequence and the second termination sequence. Use of promoters will be readily understood by one of ordinary skill the art, and the disclosures herein are not intended to be limiting, but rather, exemplary. Exemplary promoters include, for example, Pol II and Pol III promoters. In one aspect, one or both of the first promoter and second promoter may be an RNA Polymerase III (Pol II) promoter, for example one selected from Es (212 bp; GenBank Accession No. J04617.1, nucleotides 397 to 608), SV40 promoter (Sv) (269 bp; GenBank Accession No. MT086573.1, nucleotides 4403 to 4671), Elongation factor-1α promoter (nucleotides 380 to 1560; GenBank Accession No. J04617), phosphoglycerate kinase (PGK) promoter, myelin basic protein promoter, fibrillary acidic protein (GFAP) promoter, ubiquitin C promoter, MSCV (Murine Stem Cell Virus) Promoter, CAG (CAGGS) Promoter, Tetracycline-inducible Promoter, MND (synthetic) promoter, spleen focus-forming virus (SFFV) promoter, a tissue specific promoter such as thyroxine-binding globulin (TBG) promoter, and combinations thereof. In one aspect, the first promoter and second promoter are different promoters. In one aspect, one or both of the first promoter and the second promoter may be an RNA Polymerase III (Pol III) promoter. Exemplary promoters include, for example, H1, U3/U6 small nuclear RNA (snRNA) promoter, 7SK, and combinations thereof. In one aspect, where the nucleic acid sequence comprises a noncoding sequence, the corresponding promoter may comprise a Pol III promoter. See, for example, Gao Z, Herrera-Carrillo E, Berkhout B. RNA Polymerase II Activity of Type 3 Pol III Promoters. Mol Ther Nucleic Acids. 2018 Sep. 7; 12:135-145. doi: 10.1016/j.omtn.2018.05.001. Epub 2018 May 8. PMID: 30195753. www.ncbi.nlm.nih.gov/pmc/articles/PMC6023835/and Kor S D, Chowdhury N, Keot A K, Yogendra K, Chikkaputtaiah C, Sudhakar Reddy P. RNA Pol III promoters-key players in precisely targeted plant genome editing. Front Genet. 2023 Jan. 4; 13:989199. doi: 10.3389/fgene.2022.989199. PMCID: PMC9845283. www.ncbi.nlm.nih.gov/pmc/articles/PMC9845283/.

Nucleic Acid Sequences

The dual-cassette vectors may comprise at least two nucleic acid sequences. In one aspect, the first nucleic acid sequence may comprise one or both of a coding sequence and a non-coding sequence. In one aspect, the second nucleic acid sequence may comprise one or both of a coding sequence and a non-coding sequence. The first nucleic acid sequence and second nucleic acid sequence may be different sequences, each delivering a unique product, or a similar, but different product. In one aspect, the first nucleic acid sequence and/or second nucleic acid sequence may comprise a transgene. In a further aspect, one or both of the first nucleic acid sequence and the second nucleic acid sequence may comprise a therapeutic transgene, such transgene being configured to provide a therapeutic transcript or protein upon transcription or translation.

In a further aspect, one or both of the first nucleic acid sequence and the second nucleic acid sequence may comprise a sequence encoding a detectable marker. Exemplary detectable markers include, for example, GFP (Green Fluorescent Protein), RFP (Red Fluorescent Protein), YFP (Yellow Fluorescent Protein), BFP (Blue Fluorescent Protein), mCherry (mCherry is a red fluorescent protein derived from Discosoma sp.), CFP (Cyan Fluorescent Protein), Luciferase, Beta-Galactosidase (LacZ), and combinations thereof. The first and second nucleic acid sequences may encode for different detectable markers.

In one aspect, the dual-cassette vectors disclosed herein may be used to deliver component parts of a CRISPR/cas systems, for example, CRISPR-Cas systems that employ Cas9, Cas1, and Cas2, including those described in U.S. Patent Application 2021/0017509. For example, the first nucleic acid sequence of the disclosed dual-cassette vectors may comprise, consist of, or consist essentially of a CRISPR RNAs (crRNA) sequence and the second nucleic acid sequence may comprise, consist of, or consist essentially of a trans-activating CRISPR RNAs (tracrRNA) sequence, or vice versa. For example, co-expression of Cas9 and a related sgRNA in the same vector, for example an AAV vector, will enhance editing efficiency as both component will be expressed within the same transduced cell.

Termination Sequence

The TUs of the disclosed dual-cassette vectors may comprise termination sequences, such termination sequences being located downstream from the corresponding promoter and nucleic acid sequence. In one aspect, the first termination sequence is located downstream from the first promoter and the first nucleic acid sequence. In one aspect, the second termination sequence is located downstream from the second promoter and the second nucleic acid sequence. In one aspect, the first termination sequence and/or second termination sequence may be selected from a polyadenylation (poly(A)) sequence (or poly-T (thymine) sequence. The first or second termination sequence may comprises, for example, a signal sequence selected from AAUAAA, AUUAAA, UAUAAA, AGUAAA, AAGAAA, AAUAUA, AAUACA, CAUAAA, GAUAAA, CAUAAA, GAUAAA, AAUGAA, IJLJUAAA, ACU AAA, AAUAGA, AAAAAG, AAAACA, GGGGCU, and combinations thereof. In one aspect, one or both of the first termination sequence and second termination sequence may comprise a poly-T sequence, wherein the poly-T sequence comprises about 15 nucleotides to about 50 nucleotides in length, or from about 20 nucleotides to about 45 nucleotides in length, or from about 25 nucleotides to about 35 nucleotides in length, or about 30 nucleotides in length. In some aspects, the poly(T) sequence can be 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length. In one aspect, the termination sequence may be a Poly-T tail for expression of a nucleic acid sequence encoding for an snRNA or a small guide RNA (sgRNA).

cC Element

As described below, Applicant found that the disclosed cC elements provide for enhanced expression as compared to dual-cassette vectors that do not contain a cC element. The cC element may be positioned, for example, in a sense orientation downstream from the first TU, and upstream from the second TU. In other aspects, the cC element may be positioned in an antisense (reverse) orientation downstream from said first TU, and upstream from said second TU. In this aspect, the reverse/antisense cC element may be referred to as “cCrev”.

In one aspect, the cC element may comprise a 120 bp sequence corresponding to GenBank Accession No. X03922.1, -50pb to +70 bp), having the sequence TAGGCGTAGT CGGTGGGAGG TCTATATAAG CAGAGCTCGT TTAGTGAACC GTCAGATCGC CTGGAGACGC CATCCACGCT GTTTTGACCT CCATAGAAGA CACCGGGACC GATCCAGCCT. (SEQ ID NO: 1). In one aspect, the cC element may comprise at least 75%, or at least 80%, or at least 85%, or at least 90%, or at least 91%, or at least 92%, or at least 93%, or at least 94%, or at least 95%, or at least 96%, or at least 97%, or at least 98%, or at least 99% sequence identity to SEQ ID NO: 1.

In one aspect, the cC element is positioned in a reverse orientation for dual-cassette expression. That is, where two transcription units (TUs) exist on a vector, the cC element may be in a reverse orientation between the two TUs. In a further aspect, where the vector comprises a single cassette (a single TU), the cC element may be positioned in the forward orientation and located upstream or downstream from the TU.

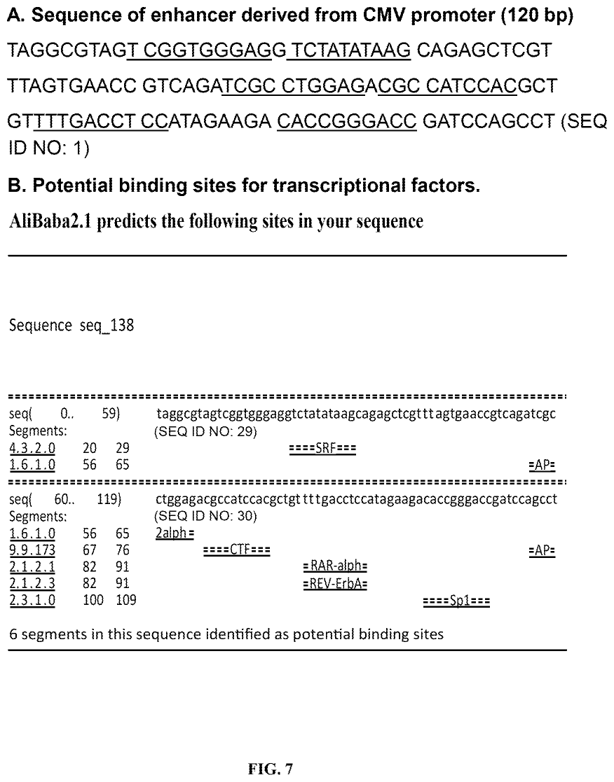

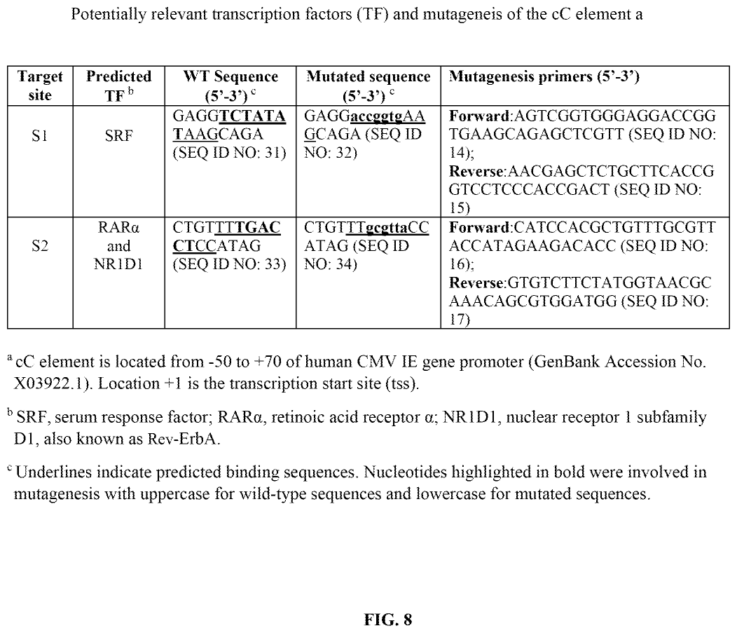

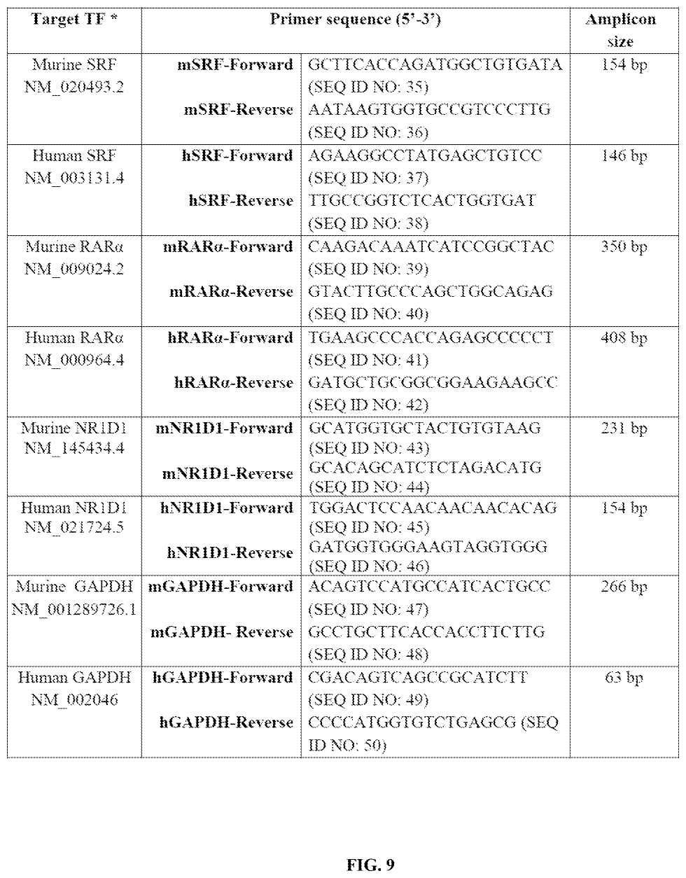

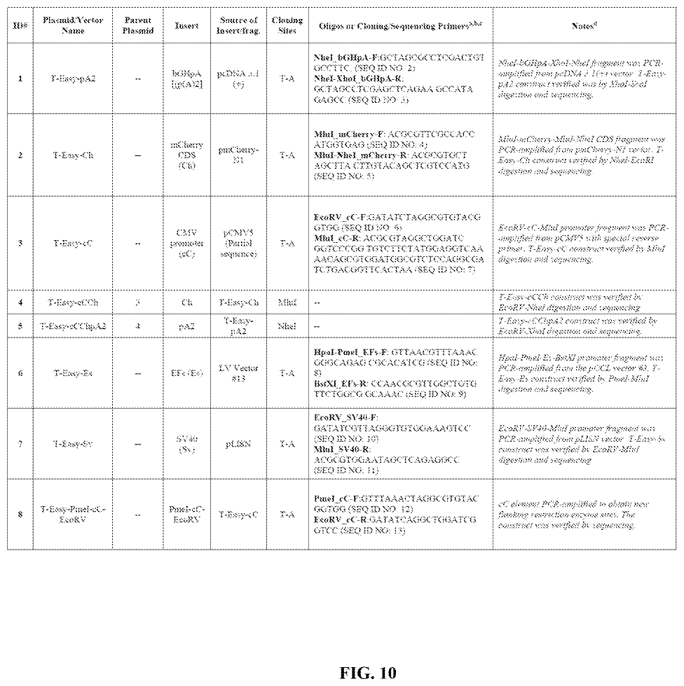

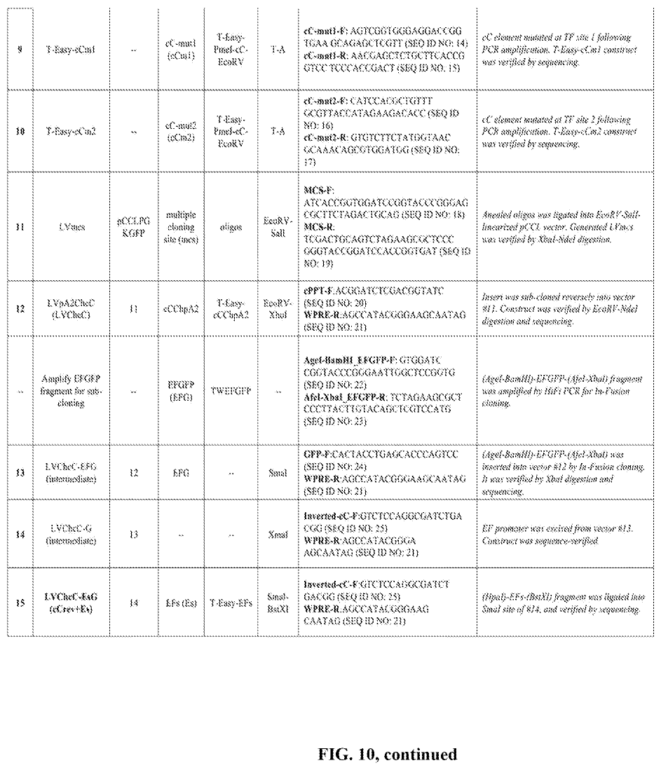

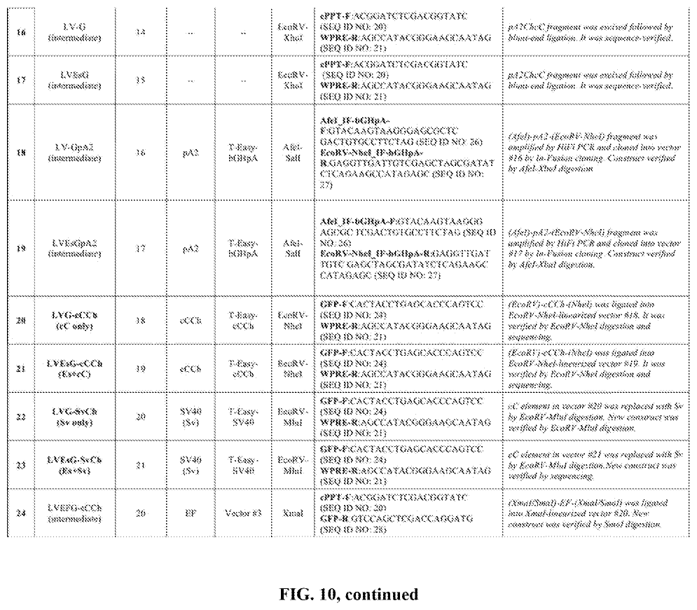

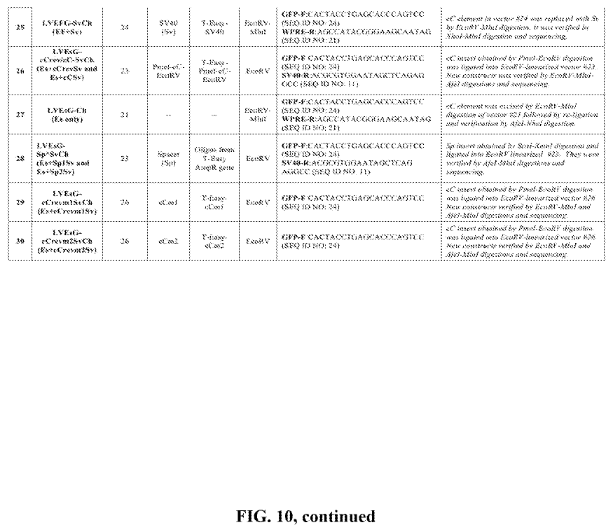

In one aspect, the cC element may comprise one or more responsive domain sites. The responsive domain sites may be, for example, those of the following sequences: “51” corresponding to 5′-TCTATATAAG-3′ (−30 to −21) (SEQ ID NO: 51, antisense “51” corresponding to 5′-CTTATATAGA-3′(SEQ ID NO: 52), “S2” corresponding to 5′-TTTGACCTCC (+33 to +42) (SEQ ID NO: 53), antisense “S2” corresponding to 5′-GGAGGTCAAA-3′(SEQ ID NO: 54). In other aspects, the cC element may comprise one or more of the underlined sequences of FIG. 7 representing a confirmed or potential transcription binding site.

In one aspect, the cC element comprises at least one, or at least two, or at least three, or four responsive domain sites selected from 5′-TCTATATAAG-3′ (−30 to −21) (SEQ ID NO: 51), antisense S1=5′-CTTATATAGA-3′ (SEQ ID NO: 52); S2=5′-TTTGACCTCC (+33 to +42) (SEQ ID NO: 53); antisense 5′-GGAGGTCAAA-3′ (SEQ ID NO: 54). In one aspect, the cC element comprises a human cytomegalovirus (CMV) promoter/exon 1 fragment, i.e., a fragment that includes sequence from both promoter (−50) and the exon 1 region (+70). In other aspects, the cC sequence may comprise 1, or 2, or 3, or 4, or 5, or 6, or 7, or 8, or 9, or 10, or 11, or 12, or 13, or 14, or 15, or 16, or 17, or 18, or 19, or 20 base pairs upstream from the tss (0), or 1, or 2, or 3, or 4, or 5, or 6, or 7, or 8, or 9, or 10, or 11, or 12, or 13, or 14, or 15, or 16, or 17, or 18, or 19, or 20 base pairs downstream from the tss (0). The cC element may further have less than 100% sequence identity to SEQ ID NO: 1, determination of operable variants of the cC element being within the skill of one of ordinary skill in the art.

The described dual-cassette vectors may be used for a variety of purposes. For example, in one aspect, disclosed is a method of treating an individual having a disease or condition, comprising one or both of administering the dual-cassette vector described herein to an individual in need thereof, the administration being an in vivo administration of the dual-cassette vector, or administering a cell to an individual, wherein the cell has been contacted with said dual-cassette vector in vitro. The disease or condition may be, for example, a cancer. In such aspect, the first nucleic acid and second nucleic acid may encode for one or both of a chimeric antigen receptor (CAR) and a cytokine, for treatment of the disease or cancer. In one aspect, the therapeutic use of the dual-cassette vector may comprise administering a vector wherein the first nucleic acid and/or second nucleic acid each encode for a CRISPR/cas protein or a sgRNA, the CRISPR/ca and sgRNA being complementary to deliver a therapeutic effect. In other aspects, the therapeutic use may comprise administering a dual-cassette vector wherein one or both of the first nucleic acid and the second nucleic acid may each encode for a portion or a subunit of a multimeric protein which, following express, may be assembled or may self-assemble into a therapeutic protein. Engineering of a CRISPR/Cas-mediated dual-cassette expression to provide co-expression of CRISPR/cas9 and sgRNA from two TUs within the same vector construct, allowing for improved editing efficiency. In other aspects, two TUs may be expressed by two promoters with one providing a therapeutic protein and the other providing a biological tracking/imaging element, or co-expression of multimeric proteins for industrial production of recombinant proteins in cell lines.

In one aspect, the disease or condition may be one which is characterized by a deficiency in expression of a gene. In such instances, the dual-cassette vector may comprise a first nucleic acid sequence and/or a second nucleic acid sequence that encodes for one or more copies of the gene which is deficient in the individual or for a disease-modifier gene.

Other exemplary uses include hematopoietic stem/progenitor cell (HSPC) gene therapy using an LV-mediated autologous transplantation for treatment of a genetic disease and transfusion of cells which have been engineered using the disclosed dual-cassette vectors. For example, the boosting effects of the cC element in single-cassette TUs is particularly useful in HSPC gene therapy (blood-lineages).

In one aspect, the cells that may be targeted using the disclosed dual-cassette vectors may be a cell that expresses, or has increased expression relative to other cell types, a gene selected from SRF, RAR-alpha, NR1D1, AP-2alpha, CTF, and sp1. That is, such cell may be a cell expressing a protein that binds to a transcription element binding site in the cC element as described herein. Following a contacting step in which the dual-cassette vector is integrated into the cell in vitro, the cell can then be administered to an individual in need thereof. In one aspect, the cell may be one that has been obtained from the individual in need of treatment, or from a donor individual for transplant into the individual in need of treatment. In one aspect, the cell is a hematopoietic stem cell.

In some aspects, the dual-cassette vectors may be provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more dual-cassette vectors provided herein in combination with one or more additional active agents may be provided, or separate pharmaceutical compositions containing a dual-cassette vector as provided herein and additional therapeutic agents may be provided. The kit may also contain separate doses for serial or sequential administration. The kit may optionally contain one or more diagnostic tools and instructions for use. The kit may contain suitable delivery devices, e.g., syringes, and the like, along with instructions for administering the dual-cassette vectors and any other therapeutic agent. The kit may optionally contain instructions for storage, reconstitution (if applicable), and administration of any or all therapeutic agents included. The kits may include a plurality of containers reflecting the number of administrations to be given to a subject. Likewise, the above-described kits may be formulated for use in in vitro systems such as cell based systems which may be useful for research purposes or for preparation of cells that may be administered to an individual in need thereof.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Lentiviral-vectors for dual-cassette expression of green fluorescent protein (GFP) and mCherry in uni- or bidirectional architectures were engineered using short-version (Es) of elongation factor 1α (EF) promoter and simian virus 40 promoter (Sv). The regulatory function of a core fragment (cC) from human cytomegalovirus (CMV) promoter was investigated with cell-lineage specificity in NIH3T3 (fibroblast) and hematopoietic cell lines U937 (monocyte/macrophage), LCL (lymphoid), DAMI (megakaryocyte), and MEL (erythroid).

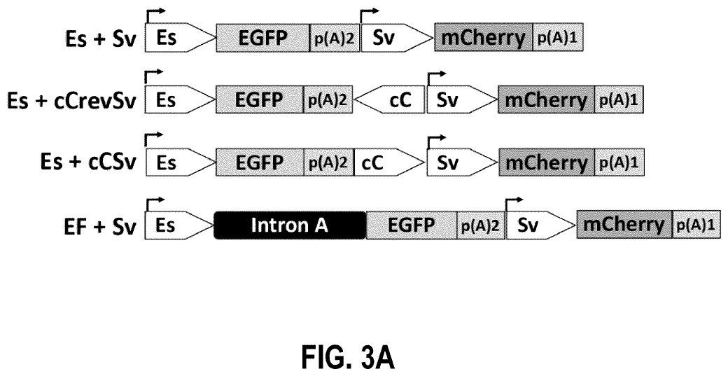

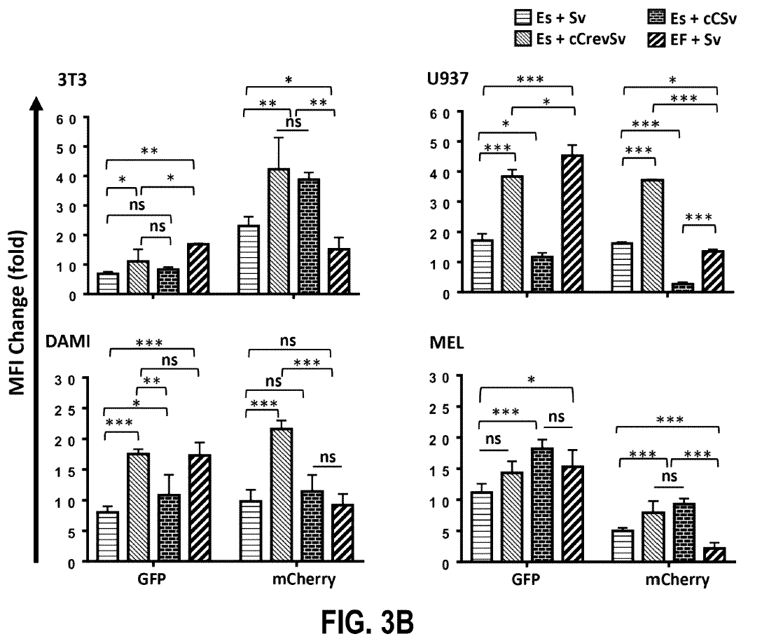

Results. The cC element in reverse-orientation not only boosted upstream Es promoter to levels comparable to full-length EF in DAMI, U937 and 3T3 cells, but also blocked the suppression of downstream Sv promoter by Es in U937 and 3T3 cells with further improved Sv activity in DAMI cells. Such lineage-restricted upregulation is likely attributed by two protein-binding domains of cC and diverse expression of related factors in different cell types for enhancer and terminator activities, but not spacing function. The cC element in forward-orientation improved expression from both TUs in MEL cells and from downstream Sv in 3T3 cells (as seen in FIG. 3)

Promoters for human cytomegalovirus major immediate-early protein (CMV), the simian virus 40 immediate-early protein (SV40) and the short version (Es) of human elongation factor 1α (EF1a) have been widely used for transgene expressions. The advantages of their comparatively smaller size have made them more applicable in dual-cassette vectors and size-restricted adeno-associated viral vectors (AAV), although they are not the strongest of constitutive promoters and viral promoters are prone to methylation. Conversely, by combining desirable enhancer and promoter elements coordinatively, hybrid/synthetic promoters, even though inevitably larger, can achieve more controlled expressions that are either robust and sustainable avoiding silencing, or tissue restricted or responsive to external stimuli. Moreover, reducing the size of such chimeric enhancer/promoters without sacrificing appropriate features has become highly desirable for the expression of multicistronic transgenes in vector design. Thus, the disclosed cC elements may be advantageously used in single-cassette TUs as compared to vectors using a full-length hybrid CAG promoter (−1.7Kb), which is significantly larger.

The full-length CMV promoter is the most commonly used and versatile regulatory element with a TATA-box-containing promoter region and an enhancer region from −550 bp to −39 bp relative to the transcription start site (tss) (GenBank Accession No. X03922.1). Multiple fragments with different sizes from the enhancer region have been utilized to boost both ubiquitous and cell-specific promoters, including CBh and CAG as well as cardiomyocyte-, alveolar epithelia- or neuron-specific promoters. Moreover, a 120-bp derivate of the core CMV promoter-extended region (cC element), containing nucleotides of −50 bp to +70 bp, has been employed as a minimal promoter for inducible transgene expression in mammalian cells in vitro. However, no other functions have been explored for this small-size cC element.

In this study, use of the small-size 120 bp cC element, in cooperation with the short but weak Es (212 bp; GenBank Accession No. J04617.1, nucleotides 397 to 608) and SV40 promoter (Sv) (269 bp; GenBank Accession No. MT086573.1, nucleotides 4403 to 4671), for modulating transcription interference between two TUs in dual-cassette LVs and for simultaneously providing relatively small cumulative size of regulatory elements which are often associated with optimal titers or required for vectors with size restriction (such as AAV) was investigated. Expression levels of green fluorescent protein (GFP) and a variant of red fluorescent protein (mCherry) driven from two independent promoters were evaluated in cells from different hematopoietic lineages, including cell lines of macrophage/monocytic (U937), megakaryocytic (DAMI), lymphoid (LCL) and erythroid lineages (MEL). Distinguished from its promoter activity, a novel function of cC element was documented that, when positioned in reverse orientation proximal to the downstream TU, cC could not only block promoter interference but also increase both upstream and downstream transgene expression with lineage-restriction. The modes of actions were also studied by site-directed mutagenesis and RT-qPCR, identifying two new protein-binding domains that may provide transcription factors (TFs)-associated enhancer function as well as protein-binding terminator function, but not spacer function. These findings establish new promoter-pairs, small in size but efficient in strength, for dual-cassette expression especially in hematopoietic cell types that could contribute significantly to the field of hematopoietic cell-mediated gene- or cancer-therapy.

Material and Methods

Primers and Oligonucleotides

Primers and oligonucleotide sequences were designed in-house with SnapGene Viewer software (GSL Biotech, Chicago, IL) and synthesized by Integrated DNA Technologies Inc (Coralville, IA).

Construction of Plasmids and Vectors

A self-inactivating lentiviral vector expressing GFP from human phosphoglycerate kinase promoter (PGK) (pCCLhPGKGFP) was a kind gift from Dr. Luigi Naldini (San Raffaele University, Milan, ITALY), which served as the backbone of all vectors. To avoid excessive manipulation of the parental LV (transfer), assembly of individual components of the expression cassettes was mostly done in the pGEM® T-Easy (T-Easy) cloning vector system (Promega, Madison, WI) prior to cloning into the LV vector. LV vectors were, however, on some occasions directly manipulated to add or remove components.

For dual-cassette expression, a bovine growth hormone (bGH) poly(A) signal sequence (nucleotides 791.1015; GenBank Accession No. JQ624676.1) was used and termed as p(A)2, while the SV40 poly(A) sequence was on the 3′LTR of LV backbone and termed as p(A)1. The EF-GFP cassette was generated when a 1181 bp fragment comprising the EF promoter (nucleotides 380 to 1560; GenBank Accession No. J04617) and enhanced GFP (nucleotides 6895 to 7611; GenBank Accession No. MN517551.1) was PCR-amplified from an in-house TW-EFGFP vector previously constructed 27. The mCherry (Ch) coding sequence (CDS) (nucleotides 904 to 1611; GenBank Accession No. LC311025) was obtained from Dr. Roger Y. Tsien (University of California San Diego, CA, USA). For the construction of spacer-containing vectors (LVEsG-Sp*SyCh), a 119 bp fragment of the ampicillin-resistant CDS (1337 to 2197 in the backbone of T-Easy vector) was excised at the ScaI-Xmnl sites and inserted as single (Sp1) and tandem repeats (Sp2) into EcoRV-site LVEsG-SvCh vector.

A detailed description for the construction of various plasmids and LV-based vectors used in this work is shown in the figures and described herein. Restriction enzymes used were obtained from New England Biologics Inc. (Ipswich, MA), together with In-fusion HD Cloning system (Takara Bio USA Inc., Mountain View, CA, USA) in some cases.

Lentiviral Vector Production

Infectious viral particles of each transfer vector were produced in human embryonic kidney (HEK) 293T cells (ATCC, Manassas, VA) using the third generation (four-plasmid) split packaging system by the calcium phosphate precipitation method as previously described. In brief, this involved transient transfection of 293T cells with a transfer vector plasmid together with three helper plasmids—pMDL.g/pRRE, pMD2.VSVG and pIL.VV01(Rev). A mixture of the four plasmids in appropriate ratios in cold calcium chloride solution was made. HEPES-buffered saline was slowly added to the DNA mixture and mixed thoroughly to form a co-precipitate of DNA and calcium phosphate. The precipitate was slowly added to freshly seeded packaging (HEK293T) cells in tissue culture plates or flasks under constant swirling to ensure even distribution of the precipitate mixture. 12-18h after incubating (at 5% CO2, 37C) the cells-precipitate mixture the transfection medium was replaced with Gibco™ Dulbeco's Modified Eagle's Medium (DMEM) supplemented with 5% fetal bovine serum (FBS). Virus supernatant was harvested 24 hours after medium change. It was passed through 0.45 μm pore-size PES filter membranes (GE Healthcare, IL) and stored in aliquots in −80C until needed.

Cell Culture

All cell culture media were obtained from Thermo Fisher Scientific (Waltham, MA). HEK 293T, and murine erythroleukemia (MEL) and NIH-3T3 (3T3) cell lines were cultured in DMEM supplemented with 10% fetal bovine serum (FBS) (HyClone), 1% L-glutathione and 1% penicillin-streptomycin (Invitrogen). DAMI (Human megakaryocytic) cell lines were maintained in Iscove's Modified Dulbeco's Medium containing 10% FBS, and 1% penicillin-streptomycin while U937 (human monoblastic leukemia) and LCL (human lymphoblastoid) cell lines were cultured in RPMI 1640 medium containing 10% and 20% FBS, respectively 1% L-glutathione and 1% penicillin-streptomycin. Cells were sub-cultured every 3-4 days depending on confluency.

Virus Transduction

Virus transduction was done as per an in-house protocol. Briefly, adherent cells were seeded (2×10 5/well) in 24-well tissue culture plates (Corning Inc. NY) containing cell culture medium and 8 ng/mL polybrene (Hexadimethrine bromide, Sigma, MO). Cells were transduced at different multiplexity of infection (MOI) so to generate transduced population with <25% transduction efficiencies. After 12-18 hrs exposure to the virus (in 5% CO2; 37C incubator) the (transduction) vector-containing medium was carefully replaced with fresh culture medium and maintained for 14 days. Cells were then digested with Gibco™ 0.05% trypsin-EDTA and reconstituted in medium for flow cytometry. Suspension cells were seeded (5×10⁴/well) in 48-well culture plates (Corning Inc. NY) in transduction medium with various MOI. They were then incubated for 12-18 hrs after which they were centrifuged at 1200 rpm for 1 hr at room temperature. Cell pellets were reconstituted in fresh medium and maintained in culture for 14 days after which aliquots were assayed for expression determination by flow cytometry.

Flow Cytometry

Transgene expression levels were quantified using BD Canto I (Becton-Dickinson, CA) and data was processed and analyzed using FACS Diva (Becton-Dickson, CA).

Informatics and Transcription Factors Site-Specific Mutagenesis

Potential TF binding domains on the cC element were searched for using two online prediction software for mammalian transcription factors—Tfsite scan (www.ifti.org/cgi-bin/ifti/Tfsitescan.pl) and Transfac (Alibaba 2.1) (gene-regulation.com/pub/programs/alibaba2/). Predictions were evaluated and two unique domains predicted for relatively uncommon transcription regulators by each software were selected. To verify the function of the selected TF binding domains, two mutation primer pairs were generated, each designed to harbor mutation in one of the binding domains. PCR amplification of the cC fragment with the two primer pairs produced two mutated cC fragments. Each mutated fragment was first cloned into a T-Easy vector and their sequences verified. Subsequently, each fragment was inserted into an EcoRV-linearized and dephosphorylated LVEsG-SvCh vector via EcoRV-Pmel excision from T-Easy vector. The resulting fragments were sequence-verified for their required orientation.

RT-qPCR Analysis

Total RNA was isolated from cell lines in culture using the RNeasy Kit (Qiagen). RT-qPCR was carried out. After quantification with NanoDrop 1000 spectrophotometer (Thermo Scientific), 1 μg of total RNA was reverse transcribed using oligo(dT)12-18 as primer using a SuperScript™ II Reverse Transcriptase kit (Invitrogen) in a 20 μL reaction mixture. The resulting cDNA was appropriately diluted as template for quantitative PCR using iTaq Universal SYBR Green Supermix (Bio-Rad) and primers. Expression of genes of interest were acquired and analyzed using ABI 7900 (Applied Biosystems) based on the protocol provided by the manufacturer. The PCR amplification condition included 2 min at 50° C., 10 min at 95° C., followed by 40 cycles of 15 s at 95° C. and 1 min at 60° C. Glyceraldehyde 3 phosphate dehydrogenase (GAPDH) was applied as internal controls for mRNA quantification. Relative expression of mRNAs was determined by the 2—ΔΔCT method.

Statistical Analysis

Data were acquired as duplicates or triplicates in at least two repeated experiments and are presented here as mean±SD (or SEM where expressly stated). Comparison between groups were done using One-way ANOVA with Tukey's comparison test using GraphPad Prism version 7.0 for Windows (GraphPad Software, CA). P<0.05 was considered statistically significant.

Results

Relative positioning and strength of promoters affect expression interference in dual-cassette vector

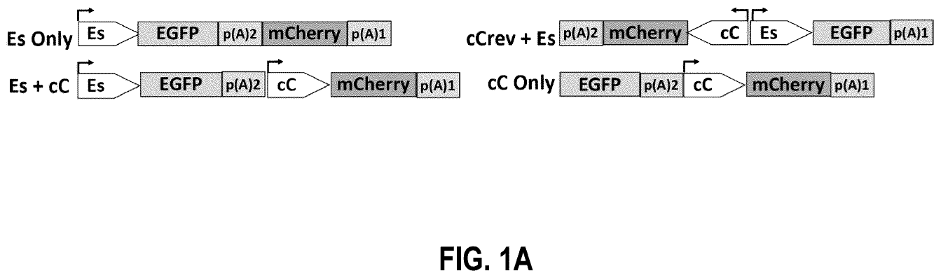

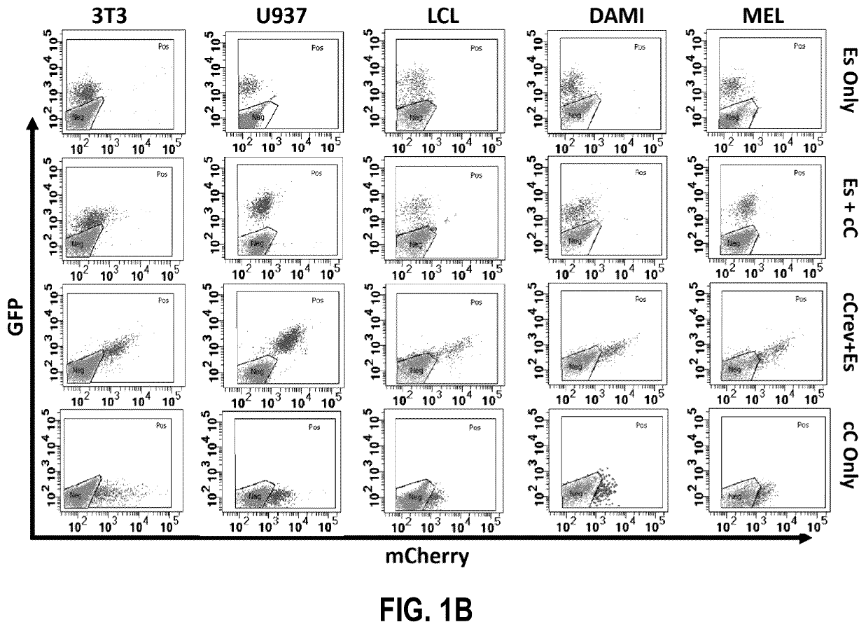

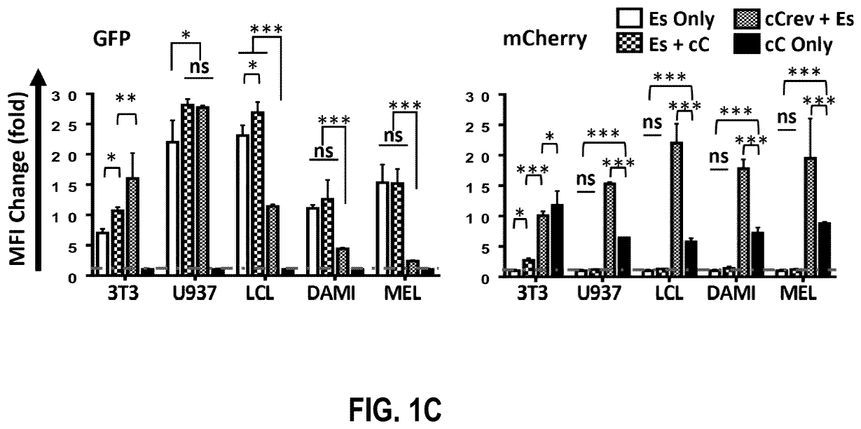

To assess the suitability of Es, cC and Sv as a promoter for transgene expression, GFP and mCherry levels from a combination of promoters in cells stably transduced with various LV vectors were tested (FIG. 1, panel A). The functionality of each promoter was validated using single-cassette control vectors in non-hematopoietic murine fibroblast NIH3T3, and hematopoietic human monocytic U937, lymphoblastic LCL, megakaryocytic DAMI and murine erythroid MEL cells by FACS analysis (FIG. 1, panel B). Without interference from adjacent TU, the mean fluorescence intensity (MFI) of GFP from Es promoter was high in both LCL and U937 cells (up to 23-fold of untransduced cells), followed by MEL (15-fold), DAMI (11-fold) and lowest in 3T3 (−7-fold) (FIG. 1, panel C). Conversely, the promoter activity of cC element was relatively higher than Es in 3T3 (−12-fold) but lower in all hematopoietic cell lines (<9-fold) (FIG. 1, panel C). When pairing Es and cC in-cis (Es+cC) in a dual-cassette LV vector, a significant increase in Es-derived GFP expression in 3T3, U937 and LCL cell lines was observed, but no change in DAMI and MEL cells, suggesting an enhancer function of cC element in monocytic, lymphoid or fibroblastic cells (FIG. 1B and FIG. 1C). On the other hand, downstream mCherry expression was completely shut down in all the hematopoietic cells while significantly reduced in 3T3 (by 77% of control), suggesting a loss in promoter activity of cC. Interestingly, when combined in divergent bidirectional LV (cCrev+Es), GFP expression was rather reduced substantially in all the hematopoietic cell lines (down to 16% of Es only) except in U937 where Es promoter activity was similarly higher than control (Es only) as that in unidirectional setting (Es+cC). On the other hand, mCherry expression from cC was significantly enhanced in all the hematopoietic cell lines (by 2.2 to 3.4-fold) compared to cC only (FIG. 1, panel C). These results show strength- and position-dependent interference (suppression) between Es- and cC-driven TUs and indicate a position-dependent enhancer activity of cC element to “dismal” Es promoter, most notably in 3T3 and U937.

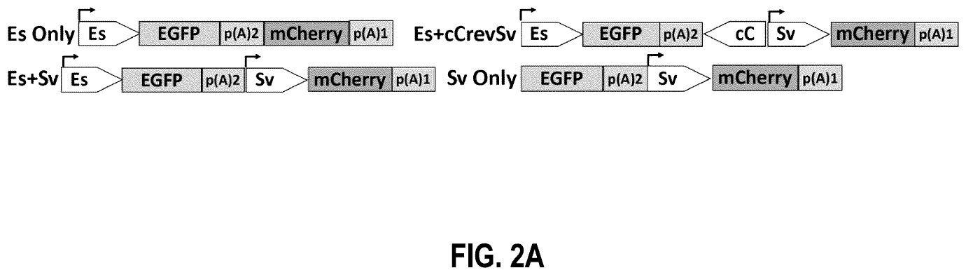

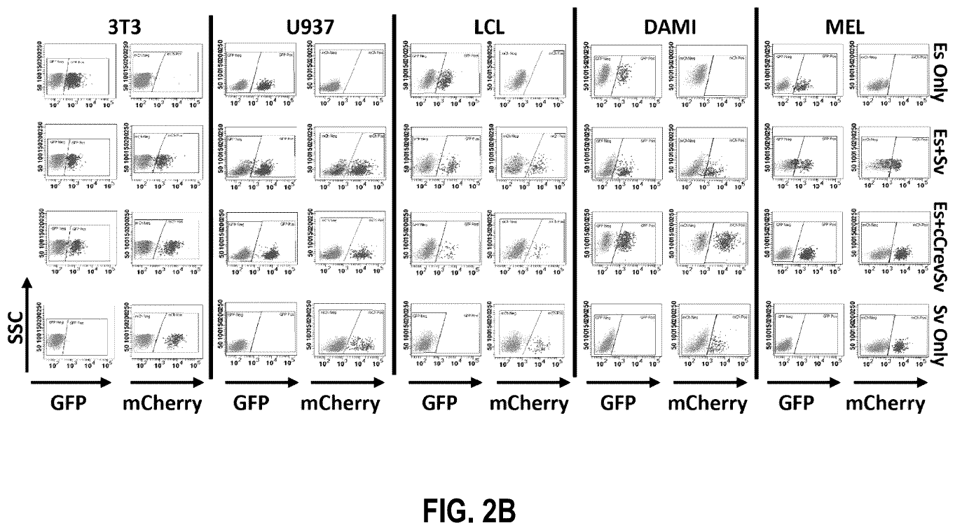

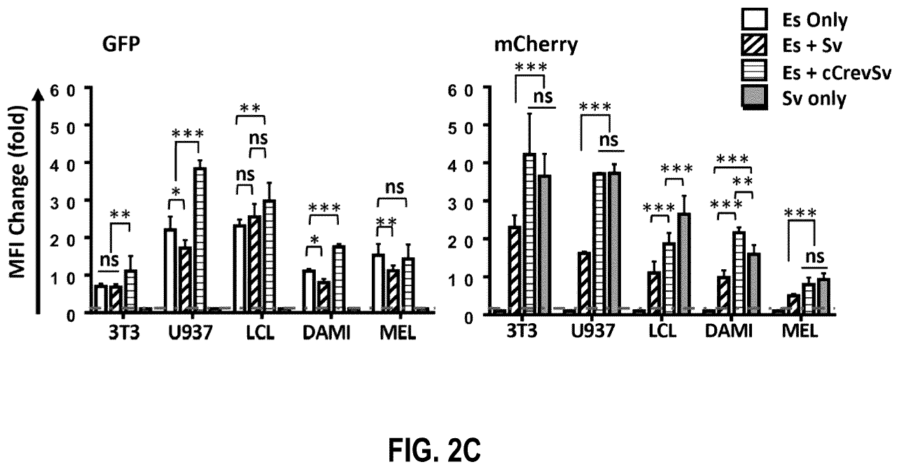

To explore whether the suppressing or enhancing effects between Es and cC is unique, the cC promoter in the unidirectional construct was replaced with Sv promoter (also small-sized, 269 bp) (FIG. 2A). In its single-cassette vector, Sv promoter exhibited higher activities than both Es and cC in all (but MEL) cell lines tested (FIG. 2, panel B and FIG. 2, panel C). In the Es+Sv construct, mCherry expression was significantly decreased in all the cell lines, suggesting again that the suppression of downstream TU by upstream Es was position-related (FIG. 2, panel B and FIG. 2, panel C). However, unlike in the Es+cC construct, GFP expression from Es promoter was either impaired by downstream Sv promoter in U937 (by 22%), DAMI (by 28%) and MEL (by 27%), or no change in 3T3 and LCL. The data demonstrated that the suppressive effect between promoters in dual-cassette vectors may be a factor of their comparative strengths and relative position, while the enhancement for upstream Es promoter activity may be restricted to the cC element and sensitive to cell types.

cC element enhances expressions of both markers in Es+Sv dual-cassette vector

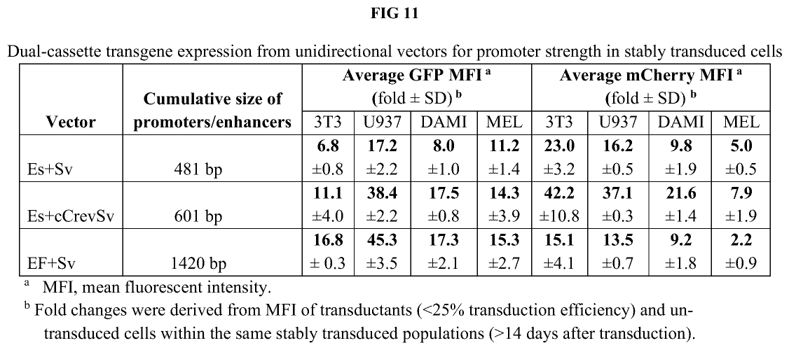

It was then hypothesized that cC may function as an enhancer element to boost expression from distal Es promoter and to reduce the reciprocal suppressive effects of both promoter in the Es+Sv vector. To test this hypothesis, the cC element was positioned between the two TUs and in reverse orientation (cCrev) to avoid any uncertainties with promoter competition between cC and Sv which may disrupt mCherry expression or cause an additive strength of a cC-Sv promoter which may suppress upstream Es (FIG. 2A). The resulting Es+cCrevSv vector had a relatively short promoter/enhancer size (cumulative of 601 bp) (FIG. 11). With this vector, GFP expression was not only higher than those with Es+Sv in U937 (from 17- to 38-fold), DAMI (from 8- to 18-fold), 3T3 (from −7- to 11-fold) and MEL cells (from 11 to 14-fold) but also further increased from those with Es only (without interference) in U937 (from 22- to 38-fold), DAMI cells (from 11- to 18-fold), and LCL cells (from 23- to 31-fold) (FIGS. 2B and 2C, FIG. 11). Significant elevation in downstream mCherry expression across all cell lines was also observed as compared to those of Es+Sv, with the highest increase detected in U937 cells (from 16-fold to 37-fold) followed by DAMI (from 10 to 22), 3T3 (from 23 to 42), LCL (from 11 to 19) and MEL cells (from 5 to 8). Such levels were either even higher than baseline SV40 promoter activity (Sv only) as in DAMI cells (1.4-fold), or comparable to baseline levels (without suppression) in all other cells except for LCL where mCherry expression was still significantly lower than baseline (FIG. 2C). The results showed that cC element could function as an enhancer in the Es/Sv dual-cassette construct to improve expression in 3T3 and all blood cell types tested.

Applicant also evaluated potential orientation specificity of cC element (forward vs. reverse) and the efficiency of dual-cassette expression from Es+cCrevSv as compared to those from EF+Sv (FIG. 3A, FIG. 11). When positioned at the same location in the forward orientation (Es+cCSv), the cC element introduced either significant increase (as in MEL and DAMI cells), or no enhancing activity (as in MEL cells) or even repressive effects (as in U937 cells) to Es-derived GFP expression, indicating that the enhancer function of cC to dismal Es promoter was affected by its orientation and cell types (FIG. 3B). The downstream mCherry expression was similarly increased by cC element, regardless of orientation, to the levels of baseline Sv only in MEL and 3T3 cells, implicating orientation-independent rescue of promoter suppression. Surprisingly, in U937 cells, mCherry expression was further reduced by >80% in cells transduced with vector Es+cCSv compared to those with Es+Sv, suggesting that cC element would play a role as a repressing element in monocytic lineage when arranged in forward orientation. The full-length human EF1α promoter, containing the Es (−0.2 kb) with an intron (A) enhancer (−1 kb), has been widely used as a strong/sustained cellular promoter in both preclinical and clinical applications. It was found that cCrev-enhanced GFP expression from weak Es promoter were comparable to those from full-length EF promoter for dual-cassette expression in all hematopoietic cell lines tested but not in the non-hematopoietic 3T3 cells (FIG. 3B). Vector Es+cCrevSv has much smaller cumulative size of promoter/enhancer elements than EF+Sv but exhibited similar (for upstream TU) or better (for downstream TU) promoter strength in dual-cassette expression (see FIG. 11). Thus, it was concluded that cC element in reverse orientation confers synergistic benefits in driving efficient dual-cassette expression from Es and Sv promoters in monocytic (U937), lymphoid (LCL), erythroid (MEL) and megakaryocytic (DAMI) cell lineages, with much less boosting effects when in forward orientation in all but erythroid cells.

The expression benefits of cC element are derived from its enhancer function rather than spacing effect

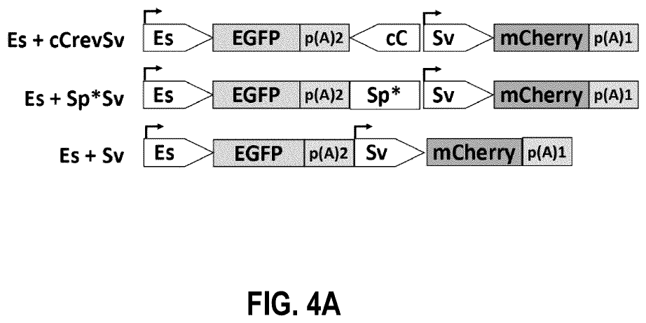

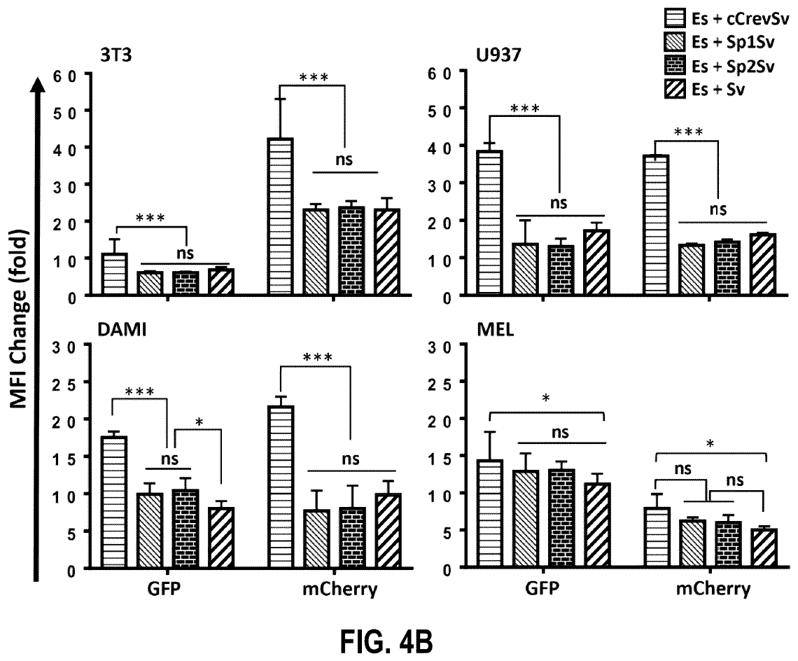

It has been reported that increasing the space between tandem expression cassettes improved dual expression of DsRed and GFP markers from CMV and PGK promoters respectively in hair follicle-derived stem cells. Whether spacer phenomenon would also contribute to the observed expression enhancement of cC was assessed by replacing it with a non-regulatory fragment obtained from the coding sequence of ampicillin resistance gene at the same (Sp1) or double (Sp2) sizes (FIG. 4A). No improvement effects were detected from any of the spacer fragments on either GFP or mCherry expression in 3T3, U937, and MEL cell lines as compared to Es+Sv (FIG. 4B). In DAMI, however, Applicant observed significant increase of upstream GFP expression to the levels comparable to Es only but less than Es+cCrevSv. It was concluded that enhancer function (protein binding), but not the spacing effect, may play a major role in cC-mediated enhancement for both upstream and downstream transgene expression.

Lineage-restrict enhancing activities of cC are correlated with transcription factor binding and relative expression of SRF and RARa/NR1D1, AP-2alpha, CTF and Sp1

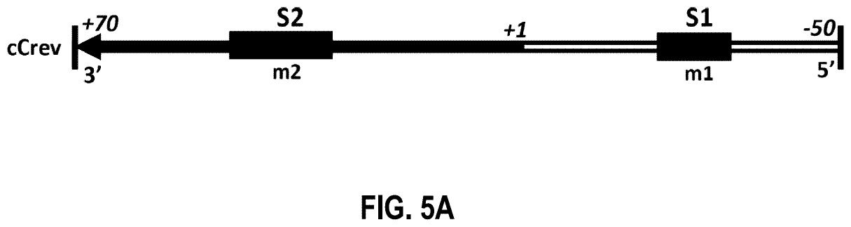

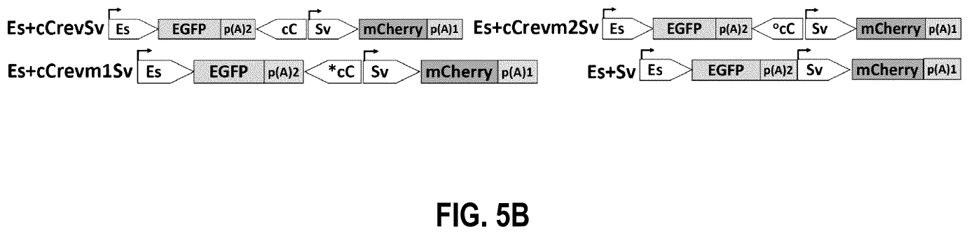

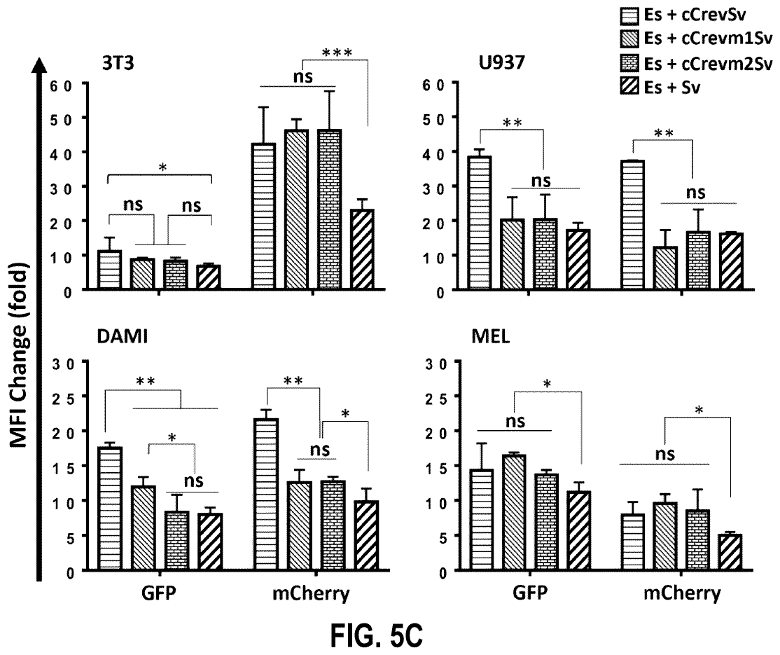

Transcriptional enhancers, also known as responsive elements, boost promoter activity by recruiting their binding factors to the transcription site so to collaborate with the promoter core initiation complex. It was then determined which transcription activator(s) may be involved in the lineage-restricted enhancer activities of cC element for both upstream Es and downstream Sv promoters. Utilizing two TF prediction algorithms, TfsiteScan and Transfac (Alibaba 2.1), two presumed binding sites (51 and S2) for non-universal TFs were identified. Serum response factor (SRF) is assumed to bind at 51 (−30 to −21), and the retinoic acid receptor alpha (RAR-α) bind at the S2 (+33 to +42) with nuclear receptor subfamily 1 group D member 1 (NR1D1) (also known as Rev-ErbA) bind at the negative strand of the same S2 site (FIG. 5A). To dissect potential significance of interaction between response domains within cC and these specific TFs, two dual-cassette LVs were constructed with cC element mutated at one of TF-binding sites by site-directed mutagenesis (FIG. 5B). Expressions from these constructs were evaluated together with Es+cCrevSv and Es+Sv in various stably transduced cells. Markedly, both mutated variants resulted in complete loss of the enhancing activities by cCrev element (as shown in Es+cCrevSv) for both upstream GFP and downstream mCherry in U937 cells, indicating the importance of these TF binding domains for promoter enhancement observed in monocytic cells (FIG. 5C). Conversely, enhanced expressions for GFP or mCherry in murine 3T3 and MEL cells (all were moderate except for mCherry in 3T3) were not significantly affected by mutations at either of the binding domains, suggesting insignificance of the tested binding sites on its enhancer activities in fibroblastic and erythroid lineages. Interestingly in DAMI cells, the boosting ability of cCrev on upstream GFP expression was partially abolished by mutation at 51 (i.e., ml) and fully diminished by mutation at S2 (m2). For mCherry MFI, both cC variants presented significant reduction from those of wild-type cC, which were still higher than mCherry expression in Es+Sv, implicating cumulative functions of 51 and S2 for enhancer activity on Sv promoter. These results suggested that both predicted TF binding domains were essential in the enhancer functions of cC element for both upstream Es and downstream Sv promoters in human monocytic (U937) and megakaryocytic (DAMI) lineages.

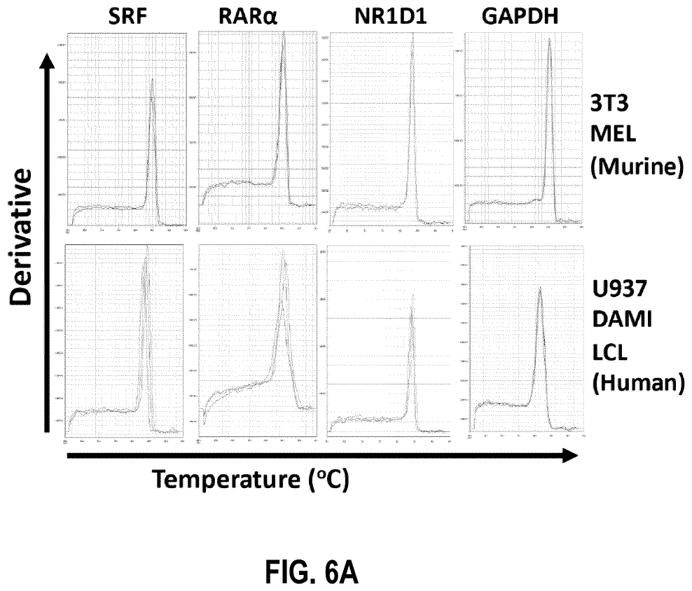

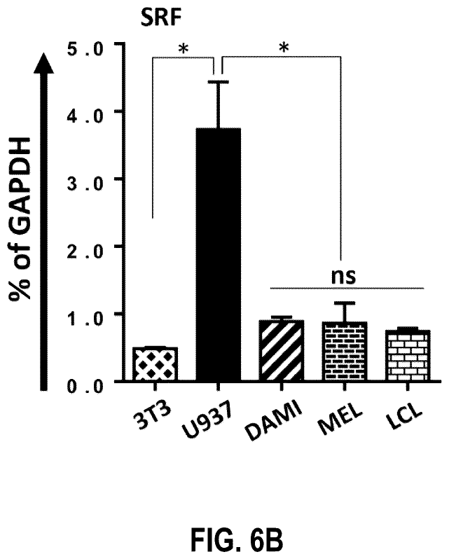

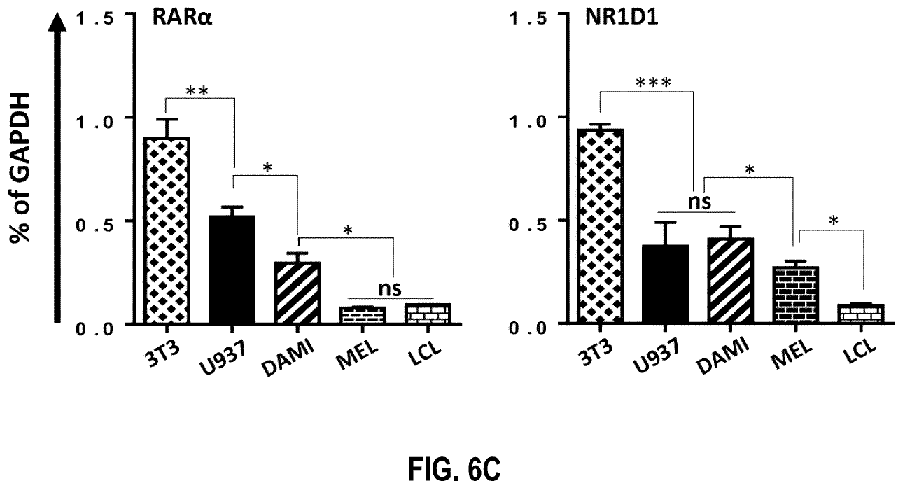

To further assess the significance of S1 and S2 on the differential enhancer effects of cC element among the various cell lineages, the relative abundance of their associated TFs, i.e., SRF, RARa and NR1D1, were quantified by RT-qPCR analysis (FIG. 6). The specificities of designed primer sets were confirmed by the dissociation curves of reactions which showed highly repeatable, single peak among different samples and cells (FIG. 6A). In 3T3 fibroblastic cells, relative expression of RARa and NR1D1 was the highest; however, their binding at S2 site seemed inconsequential as shown in mutagenesis study, suggesting other factors may contribute to the observed improvement of mCherry with Es+cCrevSV in fibroblasts (FIGS. 6B and and 6C).6C). Among blood cell lines tested, all three TFs showed the highest expression in monocytic U937 cells (FIGS. 6B and 6C). These were consistent with the observation in the mutagenesis study that S1 and S2 were essential domains for robust enhancer effects of cC element in association with both distal (Es) and proximal (Sv) promoters. Additionally, megakaryocytic DAMI cells exhibited much higher expression of RARa and NR1D1 than MEL (4- and 1.5-fold) or LCL (3- and 5-fold) but similarly low levels of S1-binding SRF among them, which coincides with the more important role of S2 domain for cC enhancer activities found by mutagenesis evaluation in DAMI cells. The lowest expression of all three TFs was observed in LCL cells where enhancer effect of cC was minimal. Taken together the data demonstrated that the differential enhancer effects of cC element among various blood cell types were closely associated with the cooperation of two specific domains within the cC element and the availability of their correspondent binding factors.

### DISCUSSION

In this study, a cell-type restricted, promoter-enhancing function of a 120 bp element (i.e., cC) from human CMV MIE promoter/exon I region (−50..+70 bp) that has not been reported previously was identified. The fragment is effective in boosting transcription from both downstream proximal promoter (Sv) and upstream distal promoter (Es) in cells stably transduced with a dual-cassette lentiviral vector (FIG. 11). Specifically, the cC element can increase Es promoter activity in all hematopoietic cells tested and reach levels as high as full-length EF promoter in monocytic U937 and megakaryocytic DAMI with orientation sensitivity. The element ascertained that downstream mCherry expression was sustained at baseline expression (without repression from upstream TU) in some cell types while exceeding baseline levels in others (DAMI). Moreover, site-specific mutagenesis studies demonstrated that responsive domains at two sites (S1 and S2) of the cC fragment play key roles in its enhancer/terminator activities detected. This observation was further supported by relative expression levels of correspondent transcription factors among different types of cells as determined by RT-qPCR. Notably, the small size of the cC element (120 bp) is a desirable feature in vector design as reducing the cumulative size of regulatory elements is often associated with optimal titers or required for vectors with size restriction (i.e., AAV). The cC element represents a potent and cell type-restricted enhancer fragment that boosts the expression and reduces promoter interference in dual-cassette vectors.

Promoter interference, defined as the influence (mostly suppressive) of one transcriptional process by the transcriptional activity of a nearby promoter, is mostly undesirable in transgene expression, although it is a natural phenomenon of gene regulation in both prokaryotic and eukaryotic genomes. Several models have been proposed for this poorly understood phenomenon that may contribute to the transcription suppression observed, mainly related to the comparative strengths of the promoters and their positioning relative to each other. The “sitting duck” model refers to the dislodging of RNA Polymerase complex from a downstream weak promoter (TU) by the transcription complex of an upstream stronger promoter due to the weak promoter's slow rate of transitioning from its transcription initiation complex. The interference in the unidirectional Es+cC construct, but not the bidirectional cCrev+Es, is consistent with this model. In single-cassette control LVs, the cC element (as a promoter) exhibited relatively ubiquitous but lower activity than upstream Es promoter in all hematopoietic cells tested, except for non-hematopoietic 3T3 cells in which cC was stronger (12-fold) than Es (7-fold) (FIG. 1C). In the Es+cC vector, the stronger upstream Es-driven TU shut down the promoter activity of the weaker downstream cC in all hematopoietic cells, but not in 3T3 cells where Es reduced cC promoter activity. In the bidirectional cCrev+Es construct, mCherry expression from cC was not only detectable but even significantly higher than baseline cC promoter activity in all hematopoietic cells tested, suggesting beneficial effect of relative positioning/orientation when both TUs proceed away from each other. However, bidirectional cCrev+Es was less optimal in viral vector design as it consistently produced very low vector titer yields as compared to unidirectional Es+cC (data not shown), consistent with observations by others for bidirectional transgene expression. Surprisingly, despite the impaired activity of cC in the unidirectional LV, cC upregulated upstream Es which suggested its potential role as an enhancer element.

Another interference model is the promoter occlusion, i.e., transcription initiation in one TU is disrupted by the arrival of an elongation complex from another TU that interrupts the assembly of DNA (promoter)-binding factors. Occlusion is often the result of a lack of adequate terminating space for the upstream TU which therefore runs into the second TU. However, due to the rate of transcription, this interference can be brief and some promoter activity from the downstream unit may be reserved. Examples could be seen in the constructs of Es+Sv (FIG. 2C) and EF+Sv (FIG. 3B). In both constructs the upstream promoter activity impaired downstream Sv promoter—the stronger EF promoter presenting more suppression—but without completely shutting down the high rate of transcription from the Sv promoter.

Finally, interference also occurs when promoters compete with each other for recruitment of binding proteins to responsive elements common to them. Depending on the abundance of a specific factor in a cell, competition may reduce availability of the factor for optimum use by two strong adjacent promoters simultaneously and therefore result in reduced expression from both TUs. The Es and Sv promoters have at least one binding site for the transcription factors Sp1 and CEBPα, while both EF and Sv have additional domains to bind AP-1, AP-2α, NF-kB, c-Jun and ETF. Thus, competition for binding proteins may contribute to the observations derived from Es/EF+Sv constructs where downstream Sv promoter activity significantly suppressed the upstream promoter in most of the hematopoietic cell lines (FIG. 3). Interference between two adjacent TUs has been commonly observed and may not be adequately explained by the simplified models above as other factors beyond the models also contribute to the phenomenon.

Inefficient transcription termination also negatively affects both upstream and downstream TUs when an elongation complex runs into and disrupts the transcription complex of the downstream adjacent unit. Poly(A) sequences are essential in vector designs to facilitate transcriptional termination of each TU and enhance their nuclear transport, mRNA stability, and translation. There are two elements believed to be important for the full functioning of poly(A) signal sequence—the hexanucleotide AAUAAA and a downstream GU or U-rich motif. Poly(A) sequence from SV40 contains both domains and proven to be one of the most efficient terminator. The bGH poly(A) sequence has no recognizable GU or U-rich motif and is considered relatively weak even though it may have other motifs that make it “sufficient” for the termination process. Relatively inefficient termination of the GFP transcription complex by the bGH poly(A) may have contributed to the interference/reduction in expression observed for both GFP and mCherry TUs in the unidirectional constructs. It may also be a factor to the lower titer observed in the bidirectional constructs when mCherry transcription complex ran into the vector 5′ LTR interrupting transcription of full-length vector RNA genome. On the other hand, the strong SV40 poly(A) located in the 3′ LTR of all the vectors ensured efficient termination of the preceding TU and would likely prevent disruption to endogenous TUs at the site of vector integration into the host genome.

Other elements without consensus motifs have also been suggested to be important for the transcription termination process, such as terminator or pause sequences. They are thought to be either bound by proteins which form DNA-protein complexes or are intrinsic DNA sequences that form structural units to affect the site delineation for final disassembly of the termination complex. Some investigators have affirmed the functional importance of terminators in the efficiency of poly(A)-dependent termination with varied sequence motifs and sizes for different genes. However, Orozco et al. and others have argued that apart from the poly(A) signal sequence no other regulatory element is required except for sufficient space to process the termination signal. Sufficient spacing was also found to benefit dual-cassette expression by reducing interference between the TUs as it allowed both TUs to effectively complete their transcription processes independently. The potential spacing effect of cC element in boosting both TUs was evaluated using scramble spacer fragments and resulted in no or minimal enhancing benefits in all cell types tested (FIG. 4B). The prospective terminator function of cC, where DNA-binding factors block the run-through of the upstream transcription complex, is likely to be associated with NR1D1 which has a single binding site (S2) on the positive strand of cCrev in Es+cCrevSv (i.e., negative strand of cC element). NR1D1 regulates transcription in several biological processes such as coordinating the circadian rhythm, metabolic pathways and cell differentiation in a heme-dependent manner in multiple cell types. For example, it has been reported to repress expression of inflammatory cytokines in macrophages and reduce the severity of peritoneal inflammation. Importantly, this factor functions as a transcriptional repressor only when binding as a homodimer to tandem repeats of its recognition sequence within promoter regions but remains inactive as a monomer. In the Es+cCrevSv construct therefore, it is speculated that NR1D1 may likely function as a DNA-binding protein to facilitate efficient termination of the GFP transcription complex, block a run-through to the downstream mCherry TU and contribute to the enhanced expression observed in U937 and DAMI cells (FIG. 2C). Such NR1D1-mediated termination benefits (for both up- and down-stream TUs) would be orientation-sensitive (strand-related), which is consistent with the loss of improvement on both TUs when cC was placed in the sense orientation as in Es+cCSv (FIG. 3B). This is also supported by the relatively high mRNA levels of NR1D1 found in U937 and DAMI cells by RT-qPCR.

The full-length CMV promoter has a known enhancer region between −550 bp and −39 bp relative to the transcription start site+1 from which different fragments (distal and proximal) have been used in the construction of hybrid promoters. For example, a 288 bp CMV enhancer fragment (−517 bp to −230 bp) has been combined with different variants of chicken β-actin promoter in CAG or CBh 2 and the fusion of a 380 bp CMV enhancer fragment (−541 bp˜−160 bp) with PDGFb promoter formed the neuron-specific CPDGFb. Whereas the 120 bp cC element (−50 bp+70 bp) studied here has only been used as a minimal promoter with very low baseline expression in combination with responsive elements to drive inducible transgene expression. For instance, the cC element functioned as the minimal promoter in combination with a tetracycline-response element for expression of transgenes in CHO cells. Recently, synthetic promoters containing the cC fragment and various regulatory elements responsive to different activated TFs were generated for ligand-induced reporter expression, resulting in high signal-to-noise ratios in response to their specific ligands in human cell lines. Applicant, however, found a novel role of the 120 bp cC fragment as an enhancer element that improves lineage-restricted dual-cassette reporter expression in monocytic (U937) and megakaryocytic (DAMI) cells, as well as enhancing lineage-restricted single-cassette TU expression in lymphoid (LCL) and monocytic (U937) cells.

Two key domains responsible for the cell-type restricted dual-enhancer function of cC fragment as demonstrated by the site-specific mutagenesis (FIG. 5) were identified and supported by the relative expression among cells of related transcription factors, SRF and RARa (FIG. 6). The predicted SRF is considered an essential transcription factor for mature myeloid cell functions and megakaryopoiesis, which was consistent with the RT-qPCR data showing (relatively) high expression of SRF in U937 cells. Mutation to the SRF binding domain in the cC element (Es+cCrevmlSv) completely abolished enhancer function of cC for both TUs in human monocytic U937 and partially reduced in megakaryocytic DAMI cells (FIG. 5C), further supporting functional significance of SRF-binding site in the observed lineage-restricted enhancer activity. The RARa protein has been reported to play important roles in myeloid differentiation. Its expression pattern in the hematopoietic cells tested, where it was most abundant in U937 and DAMI but not in MEL and LCL (FIG. 6C), was also correlated with the observation that cC element exhibited stronger enhancer effects in U937 and DAMI. Moreover, the fact that the binding sites for RARa and NR1D1 (both showed high expression in U937 and DAMI cells) were overlapped and on the opposite strands of responsive domain (S2) would likely provide a possibility of synergistic outcome from both RARa binding for enhancer activity and NR1D1 binding for terminator effect. This, in turns, may compensate for their lower expression levels than SRF in U937 cells. The mutation to the S2 site in cC (in Es+cCrevm2Sv) abrogated all enhancer benefits for both TUs in U937 and DAMI except for downstream mCherry expression in DAMI cells (FIG. 5C), demonstrating the importance of this domain on the lineage-restrictive enhancer function observed. Thus, in one aspect, the disclosed dual-cassette vectors comprise a cC element that comprises the S2 site. The results do not exclude the possibility that other co-factors may cooperate with SRF, RAR and NR1D1 at these sites, or the involvement of other untested responsive domains (especially for 3T3 cells). However, the data highlights two domains within cC elements and their correspondent factors that improve promoter activities of dual-cassette expression and substantiate the preference of such enhancer function in monocytic (U937) and megakaryocytic (DAMI) lineages.

In summary, a small fragment within the major immediate early promoter region and exon I of human CMV which functions as a terminator sequence and an enhancer element in a dual-cassette vector has been identified. This 120 bp fragment not only prevented promoter reciprocal suppression but also further increased transgene expression in 3T3 and all blood cell types tested, particularly in monocytic (U937) and megakaryocytic (DAMI) cells from two short promoters—Es and SV40 promoters. Recently, the Es promoter has shown in vivo efficacy and safety in a gene therapy clinical trial, although it is much less potent than EF promoter. Markedly the enhancer effect of cC on the Es promoter resulted in gene expression levels comparable to those from the full-length EFla promoter. The cC element significantly reduces the cumulative size of the regulatory elements in the dual-cassette vector without compromising functionality and may therefore be beneficial for vectors with limited size- or packaging-capacity. The novel design of a potent and lineage-restricted dual-cassette vector developed herein may be useful for multi-gene expression in hematopoietic cell-mediated gene and/or cancer therapy when co-expression of multiple CARs is needed.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A dual-cassette vector comprising
a. a first transcription unit (TU) comprising a first promoter, a first nucleic acid sequence, and a first termination sequence;
b. a second transcription unit (TU) downstream from said first TU; said second TU comprising a second promoter, a second nucleic acid sequence, and a second termination sequence; and
c. a cytomegalovirus (CMV) core fragment (cC element).

2. The dual-cassette vector of claim 1, wherein said vector is selected from a non-viral plasmid vector, a nanoparticle, or a virus-derived vector.

3. The dual-cassette vector of claim 1, wherein said vector is selected from a lentiviral vector, an adenoviral vector (AdV), an adeno-associated viral vector (AAV), a retroviral vector, a herpes simplex virus vector (HSV), a vaccinia virus vector, a vesicular stomatitis virus (VSV), modified vaccinia virus Ankara (MVA), arenavirus, Sendai virus, measles virus, a poxvirus vector, and combinations thereof.

4. The dual-cassette vector of claim 1, wherein said first promoter is located upstream of said first nucleic acid sequence and said first termination sequence, and wherein said second promoter is located upstream of said second nucleic acid sequence and said second termination sequence.

5. The dual-cassette vector of claim 1, wherein one or both of said first promoter and said second promoter is a Pol II or a Pol III promoter.

6. The dual-cassette vector of claim 1, wherein one or both of said first promoter and said second promoter is a Pol II promoter selected from Es (212 bp; GenBank Accession No. J04617.1, nucleotides 397 to 608), SV40 promoter (Sv) (269 bp; GenBank Accession No. MT086573.1, nucleotides 4403 to 4671), Elongation factor-1α promoter (nucleotides 380 to 1560; GenBank Accession No. J04617), phosphoglycerate kinase (PGK) promoter, myelin basic protein promoter, fibrillary acidic protein (GFAP) promoter, ubiquitin C promoter, MSCV (Murine Stem Cell Virus) Promoter, CAG (CAGGS) Promoter, Tetracycline-inducible Promoter, MND (synthetic) promoter, spleen focus-forming virus (SFFV) promoter, a tissue specific promoter such as thyroxine-binding globulin (TBG) promoter.

7. The dual-cassette vector of claim 1, wherein one or both of said first promoter and said second promoter is a Pol III promoter selected from H1, U3/U6 small nuclear RNA (snRNA) promoter, U5, H2, 5S, 7SK, and combinations thereof.

8. The dual-cassette vector of claim 1, wherein said first nucleic acid sequence and/or said second nucleic acid sequence comprises a sequence selected from a coding sequence, a non-coding sequence, a transgene, a therapeutic transgene, a detectable marker, and combinations thereof.

9. The dual-cassette vector of claim 1, wherein said first termination sequence is located downstream from said first promoter and said first nucleic acid sequence and wherein said second termination sequence is located downstream from said second promoter and said second nucleic acid sequence.

10. The dual-cassette vector of claim 1, wherein said first termination sequence and said second termination sequence is selected from a polyadenylation (poly(A)) sequence (or poly-T (thymine) sequence.

11. The dual-cassette vector of claim 1, wherein said cC element comprises a human cytomegalovirus (CMV) promoter/exon 1 fragment.

12. The dual-cassette vector of claim 1, wherein said cC element comprises at least 75%, or at least 80%, or at least 85%, or at least 90%, or at least 91%, or at least 92%, or at least 93%, or at least 94%, or at least 95%, or at least 96%, or at least 97%, or at least 98%, or at least 99% sequence identity to TAGGCGTAGT CGGTGGGAGG TCTATATAAG CAGAGCTCGT TTAGTGAACC GTCAGATCGC CTGGAGACGC CATCCACGCT GT TTTGACCT CCATAGAAGA CACCGGGACC GATCCAGCCT (SEQ ID NO: 1).

13. The dual-cassette vector of claim 1, wherein said cC element comprises TAGGCGTAGT CGGTGGGAGG TCTATATAAG CAGAGCTCGT TTAGTGAACC GTCAGATCGC CTGGAGACGC CATCCACGCT GT TTTGACCT CCATAGAAGA CACCGGGACC GATCCAGCCT (SEQ ID NO: 1).

14. The dual-cassette vector of claim 1, wherein said cC element is positioned in a sense orientation downstream from said first TU, and upstream from said second TU.

15. The dual-cassette vector of claim 1, wherein said cC element is positioned in an antisense (reverse) orientation downstream from said first TU, and upstream from said second TU.

16. A method of treating an individual having a disease or condition, comprising one or both of administering the dual-cassette vector of claim 1 to an individual in vivo, or administering a cell that has been contacted with said dual-cassette vector in vitro.

17. The method of claim 16, wherein said disease or condition is cancer or an inherited disease.

18. The method of claim 16, wherein one or both of said first nucleic acid and second nucleic acid encodes for a chimeric antigen receptor (CAR), a cytokine, and combinations thereof.

19. The method of claim 16, wherein said one or both of said first nucleic acid and second nucleic acid each encode for a CRISPR/cas protein or a sgRNA.

20. The method of claim 16, wherein said one or both of said first nucleic acid and second nucleic acid each encode for a portion or subunit of a multimeric protein.

21. The method of claim 16, wherein said disease or condition is a disease or condition characterized by a deficiency in expression of a gene with or without a known disease-modifier gene, wherein said first nucleic acid sequence or said second nucleic sequence encodes for said gene.

22. The method of claim 16, wherein said cell that has been contacted with said dual-cassette vector in vitro is a cell that expresses a gene selected from SRF, RAR-alpha, NR1D1, AP-2alpha, CTF, and sp1.

23. The method of claim 16, wherein said cell that has been contacted with said dual-cassette vector in vitro is a cell expressing a protein that binds to a binding site in said cC element.

24. The method of claim 16, wherein said cell that has been contacted with said dual-cassette vector in vitro is obtained from said individual.

25. The method of claim 16, wherein said cell that has been contacted with said dual-cassette vector in vitro is a hematopoietic/progenitor stem cell.

26. A single-cassette vector comprising
a) a single transcription unit (TU), said TU comprising a promoter, first nucleic acid sequence, and a termination sequence; and
b) a cytomegalovirus (CMV) core fragment (cC element) comprising at least 90% sequence identity to SEQ ID NO: 1.

27. The method of claim 26, wherein said cC element is in a forward orientation downstream from said transcription unit (TU).

28. The method of claim 26, wherein said cC element is in a reverse orientation downstream from said transcription unit (TU).

29. The single-cassette vector of claim 26, wherein said TU comprises an IRES or 2A-link element.
